(19) United States
(12) Patent Application Publication
(10) Pub. No.: US 2008/0255426 A1
(43) Pub. Date: Oct. 16, 2008

(54) AUTOFLUORESCENCE ENDOSCOPE SYSTEM AND LIGHT-SOURCE UNIT

(75) Inventor: Kohei IKETANI, Saitama (JP)

Correspondence Address: GREENBLUM & BERNSTEIN, P.L.C., 1950 ROLAND CLARKE PLACE, RESTON, VA, 20191, US

(73) Assignee: HOYA CORPORATION, Tokyo (JP)

(21) Appl. No.: 12/101,435

(22) Filed: Apr. 11, 2008

(30) Foreign Application Priority Data
Apr. 13, 2007 (JP) 2007-105577

(51) Int. Cl. A61B 1/06

(52) U.S. Cl. 600/180

(57) ABSTRACT

An autofluorescence endoscope system including first and second exciting light sources, an exciting-light cut-off filter, an imaging device, a light-source controller, and an imaging device driver, is provided. The first and second exciting light sources emit first and second exciting light, respectively. The wavelengths of the first and second exciting lights range in a first and second band, respectively. The first and second exciting lights make an organ autofluoresce. The wavelength in the second band is longer than that of the first band. The exciting-light cut-off filter attenuates a light component at least of the first or second band from an optical image. The imaging device captures an optical image of the subject passing the exciting-light cut-off filter. The light-source controller controls the first and second exciting light sources.

### BACKGROUND OF THE INVENTION

1. Field of the Invention

The present invention relates to an autofluorescence endoscope system for observation of an autofluorescence image of a subject.

2. Description of the Related Art

It is known that tissue autofluoresces when it is illuminated by exciting light close to a specific wavelength, such as ultraviolet light. It is also known that the degree or autofluorescence in, for examples a cancerous region, is less than that of a healthy area in an organ. An autofluorescence endoscope system providing an image to help medical examination by taking advantage of the above properties has been invented. A prior autofluorescence endoscope system provides an image enabling the estimating of the health of a region more easily than a normal image. However, better image is needed to improve diagnosis.

### SUMMARY OF THE INVENTION

Therefore, an object of the present invention is to provide an autofluorescence endoscope system that provides various kinds of images to help in diagnosis.

According to the present invention, an autofluorescence endoscope system comprising first and second exciting light sources, an exciting-light out-off filter, an imaging device, a light-source controller, and an imaging device driver, is provided. The first exciting light source emits first exciting light. The wavelength of the first exciting light ranges in a first band. The first exciting light makes an organ autofluoresce. The second exciting light source emits second exciting light. The wavelength of the second exciting light ranges in a second band. The wavelength in the second band is longer than that of the first band. The second exciting light makes an organ autofluoresce. The exciting-light cut-off filter attenuates a light component at least of the first or second bands from an optical image of a subject illuminated by the first and second exciting lights. The imaging device captures an optical image of the required subject passing the exciting-light cut-off filter. The imaging device generates an image signal corresponding to a captured optical image. The light-source controller controls the first and second exciting light sources. The imaging device driver drives the imaging device.

Further, the exciting-light cut-off filter is a trap filter which attenuates exciting light of the second hand. Or, the exciting-light cut-off filter attenuates exciting light whose wavelength is equal to or less than said second band. Or, the exciting-light cut-off filter attenuates exciting light whose wavelength is equal to or less than said first band.

According to the present invention, a light-source unit comprising first and second exciting light sources, a receiver, and a light-source controller, is provided. The light-source unit supplies light to illuminate a subject at a head end of an insertion tube of an endoscope. The first exciting light source emits first exciting light. The wavelength of the first exciting light ranges in a first band. The first exciting light makes an organ autofluoresce. The second exciting light source emits second exciting light. The wavelength of the second exciting light ranges in a second band. The wavelength of the second band is longer than that of the first band. The second exciting light makes an organ autofluoresce.

### BRIEF DESCRIPTION OF THE DRAWINGS

The objects and advantages of the present invention will be better understood from the following description, with reference to the accompanying drawings in which:

FIG. 1 is a block diagram showing the internal structure of an autofluorescence endoscope system of an embodiment of the present invention;

FIG. 2 is a block diagram showing the internal structure of a light-source unit;

FIG. 3 is a spectrograph showing spectroscopic property of the first and second exciting lights;

FIG. 4 is a spectrograph showing the transmittance of a 430 nm cut-off filter;

FIG. 5 is a spectrograph showing the transmittance of a 460 nm cut-off filter;

FIG. 6 is a spectrograph showing the transmittance of a 445 nm trap filter;

FIG. 7 is a block diagram showing the internal structure of an image-processing unit;

FIG. 8 is a so-called normal image displayed on the monitor;

FIG. 9 is a first autofluorescence image displayed on the monitor;

FIG. 10 is a first enhanced image displayed on the monitor;

FIG. 11 is a timing chart illustrating the timing used to drive the first and second exciting light sources and shutter in the first simultaneous display mode;

FIG. 12 is a first autofluorescence image and a first enhanced image simultaneously displayed on the monitor;

FIG. 13 is a timing chart illustrating the timing used to drive the first and second exciting light sources and shutter in the second simultaneous display mode;

FIG. 14 is a normal image, a first autofluorescence image, and a second enhanced image simultaneously displayed on the monitor;

FIG. 15 is a timing chart illustrating the timing used to drive the first and second exciting light sources and shutter in the third simultaneous display mode;

FIG. 16 is a second autofluorescence image displayed on the monitor;

FIG. 17 is a timing chart illustrating the timing used to drive the first and second exciting light sources and shutter in the fifth autofluorescence image mode;

FIG. 18 is a flowchart used to explain the control process of the light-source unit and the electronic endoscope carried out by the endoscope processor;

FIG. 19 is a flowchart used to explain the subroutine of the first endoscope driving process;

FIG. 20 is a first flowchart used to explain the subroutine of the second endoscope driving process;

FIG. 21 is a second flowchart used to explain the subroutine or the second endoscope driving process;

FIG. 22 is a flowchart used to explain the subroutine of the third endoscope driving process;

FIG. 23 is a flowchart used to explain the subroutine of the fourth endoscope driving process;

FIG. 24 is a flowchart used to explain the subroutine of the driving process for normal-image mode;

FIG. 25 is a flowchart used to explain the subroutine of the driving process for the first and fourth autofluorescence image modes;

FIG. 26 is a flowchart used to explain the subroutine of the driving process for the second autofluorescence image mode;

FIG. 27 is a flowchart used to explain the subroutine of the driving process for the third and sixth autofluorescence image modes;

FIG. 28 is a flowchart used to explain the subroutine of the driving process for the enhanced-image mode;

FIG. 29 is a flowchart used to explain the subroutine of the driving process for the first simultaneous display mode and the fifth autofluorescence image mode;

FIG. 30 is a flowchart used to explain the subroutine of the driving process for the second and seventh simultaneous display modes;

FIG. 31 is a flowchart used to explain the subroutine of the driving process for the third simultaneous display mode;

FIG. 32 is a flowchart used to explain the subroutine of the driving process for the fourth simultaneous display mode,

FIG. 33 is a flowchart used to explain the subroutine of the driving process for the fifth, eighth, and ninth simultaneous display modes; and

FIG. 34 is a flowchart used to explain the subroutine of the driving process for the sixth simultaneous display mode;

### DESCRIPTION OF THE PREFERRED EMBODIMENTS

The present invention is described below with reference to the embodiment shown in the drawings.

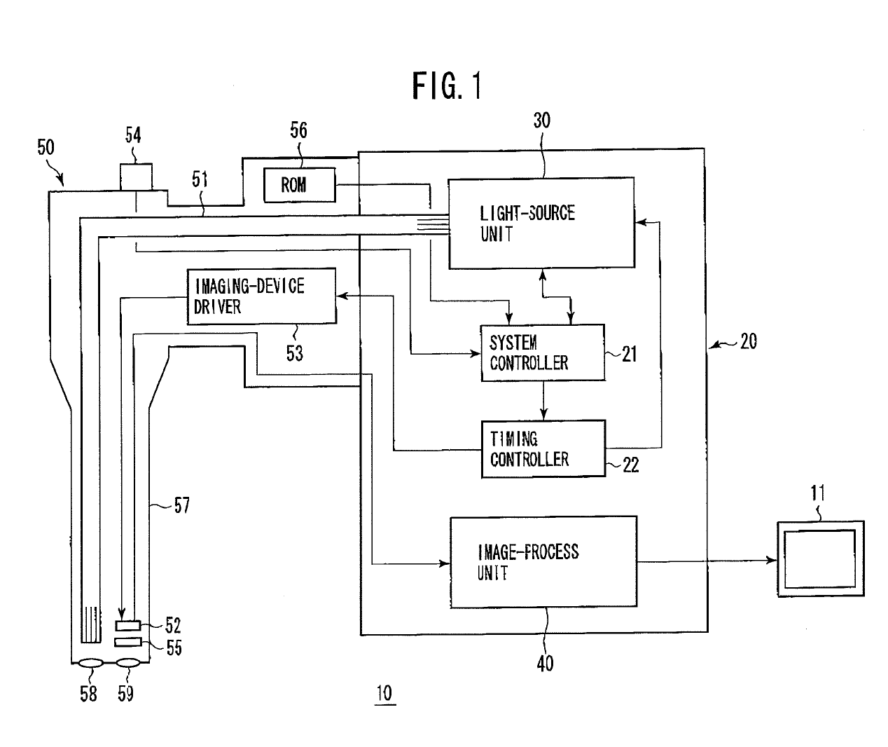

In FIG. 1, an autofluorescence endoscope system 10 comprises an endoscope processor 20, an electronic endoscope 50, and a monitor 11. The endoscope processor 20 is connected to the electronic endoscope 50 and the monitor 11.

The endoscope processor 20 emits light to illuminate a required subject. An optical image of the illuminated subject is captured by the electronic endoscope 50, and then the electronic endoscope 50 generates an image signal. The image signal is sent to the endoscope processor 20.

The endoscope processor 20 carries out predetermined signal processing on the received image signal, and then a video signal is generated. The video signal is sent to the monitor 11, where an image corresponding to the video signal is displayed.

The endoscope processor 20 comprises a light-source unit 30, an image-processing unit 40, a system controller 21, a timing controller (light-source controller) 22, and other components. As described below, the light-source unit 30 emits white light, which illuminates a desired object, and exciting light, which makes an organ autofluoresce. In addition, as described below in detail, the image-processing unit 40 carries out predetermined signal processing on the image signal.

The system controller 21 controls the operations of all components of the endoscope processor 20. The timing controller 22 times some operations of the components of the endoscope processor 20.

By connecting the endoscope processor 20 to the electronic endoscope 50, the light-source unit 30 and a light-guide 51 mounted in the electronic endoscope 50 are optically connected. In addition, by connecting the endoscope processor 20 to the electronic endoscope 50, electrical connections are made; between the image-processing unit 40 and the imaging device 52 mounted in the electronic endoscope 50; between the timing controller 22 and the imaging device via the imaging-device driver 53; and between the system controller 21 and the input block 54 mounted in the electronic endoscope 50.

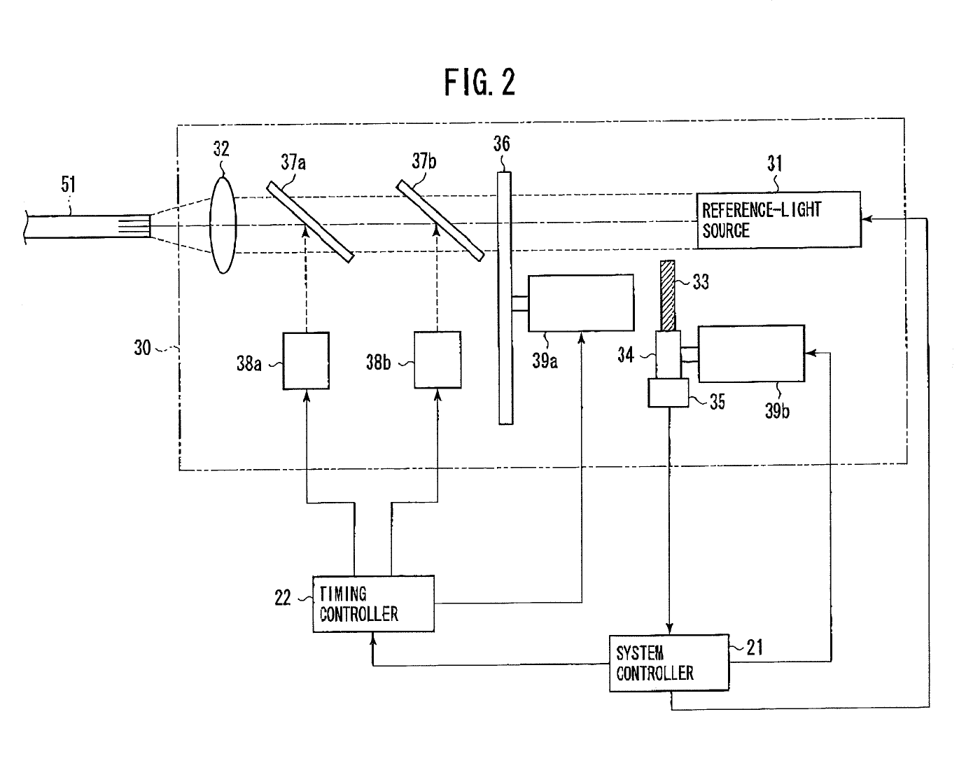

As shown in FIG. 2, the light-source unit 30 comprises a reference-light source 31, a first and second exciting light source 38a and 38b, a condenser lens 32, a light-source filter 33, a filter-driving mechanism 34, a position detector 35, a shutter 36, a first and second motor 39a and 39b, and other components.

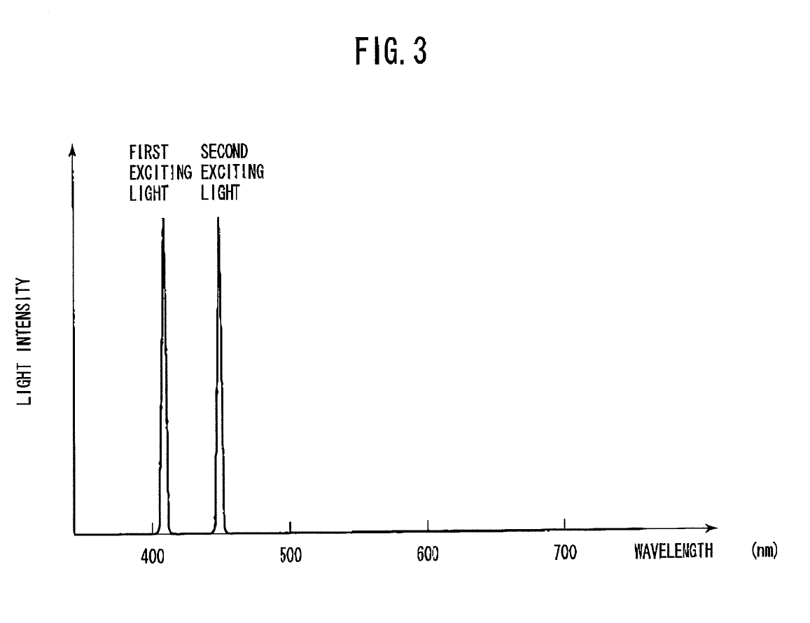

The reference-light source 31 emits white light. As shown in FIG. 3, the first and second exciting light sources 38a and 38b emit the first and second exciting lights, respectively. The first and second exciting lights are narrow-band blue light with wavelength peaks at 408 nm and 445 nm, respectively.

The shutter 36, a first and second dichroic mirror 37a and 37b, and the condenser lens 32 are mounted between the reference-light source 31 and the light guide 51. The white light emitted by the reference-light source 31 passes the first and second dichroic mirrors 37a and 37b, and is incident on the condenser lens 53. The first and second exciting lights emitted by the first and second exciting light sources 38a and 38b are reflected by the first and second dichroic mirrors 37a and 37b, respectively, and are incident on the condenser lens 53. The white light and the first and second exciting lights incident to the condenser lens 32 are condensed by the condenser lens 32, and are directed to the incident end of the light guide 51.

The first motor 39a drives the shutter so to control the passage of or block the white light. The shutter 36 is inserted into the optical path of the white light in order to block it. On the other hand, the shutter 36 is removed from the optical path of the white light when the white light is intended to reach the condenser lens 32.

The filter-driving mechanism 34 supports the light-source filter 33, and can insert and remove the light-source filter 33 into or out of the optical path of the white light emitted by the reference-light source 31. The second motor 39b drives the filter-driving mechanism 34 so that the light-source filter 33 is inserted and removed. The position detector 35 is mounted on the filter-driving mechanism 34 to detect the position of the light-source filter 33.

The light-source filter 33 attenuates the blue light component and passes green and red light components. Accordingly, as the light-source filter 33 is inserted into the optical path of the white light, green and red light components of the white light emitted by the reference-light source 31 are directed onto the incident end of the light guide 51. On the other hand, when the light-source filter 33 is removed from the optical path, the white light also arrives at the incident end.

The first and second exciting light source 38a and 38b, and the first motor 39a are connected to the timing controller 22. The timing on and off of the first and the second exciting light source 38a and 38b, and the passing and blocking of the white light by the shutter 36 is controlled by the timing controller 22.

The reference-light source 31, the second motor 39b, the position detector 35, and the timing controller 22 are connected to the system controller 21. The system controller 21 controls the timing on and off of the reference-light source 31 and some operations of the timing controller 22. In addition, the system controller 21 receives location information of the light-source filter 33 detected by the position detector 35, and controls an operation of the second motor 39b based on the detected location of the light-source filter 33.

The autofluorescence endoscope system 10 has several observation modes. As described later, turning on and off of the first and second exciting light source 38a and 38b, blocking and passing of white light by the shutter 36, and insertion and removal of the light-source filter 33 vary according to the operation mode selected for observation.

Next, the structure of the electronic endoscope 50 is explained in detail. As shown in FIG. 1, the electronic endoscope 50 comprises the light guide 51, the imaging device 52, the imaging-device driver 53, the input block 54, an exciting-light cut off filter 55, a ROM, and other components.

The light guide 51 is a bundle of optical fibers, of which one end is mounted in a connector (not depicted) which connects the electronic endoscope 50 to the endoscope processor 20, and the other end, hereinafter referred to as an exit end, is mounted in the head end of the insert tube 57 of the electronic endoscope 50. As described above, the white light and the first and second exciting lights emitted by the light-source unit 30 are incident on the incident end of the light guide 51. The light is transmitted to the exit end. The light transmitted to the exit and illuminates a peripheral area near the head end of the insert tube 57 through a diffuser lens 58.

At the head end of the insert tube 57, an object lens 59, the exciting-light cut-off filter 55, and the imaging device are also mounted. The exciting-light out-off filter 55 is arranged between the object lens 59 and the imaging device 52.

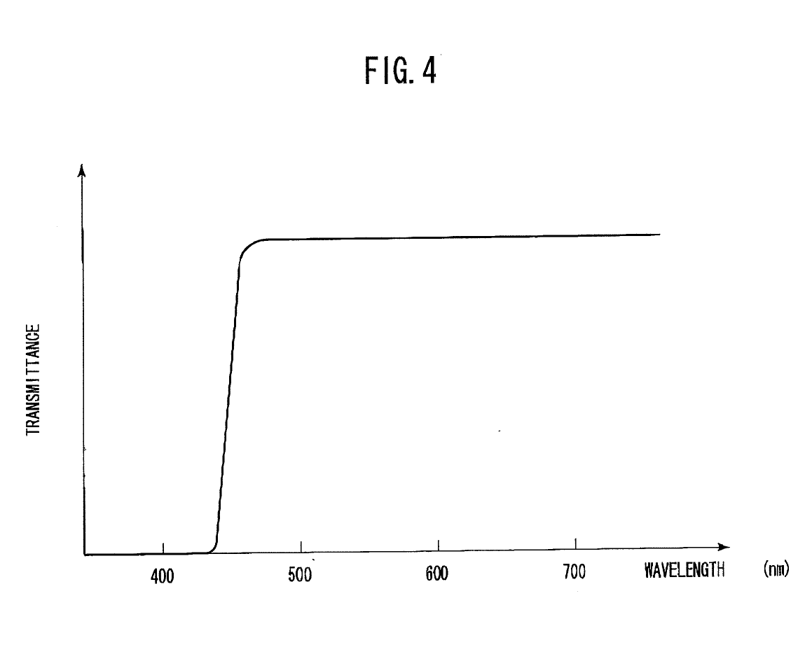

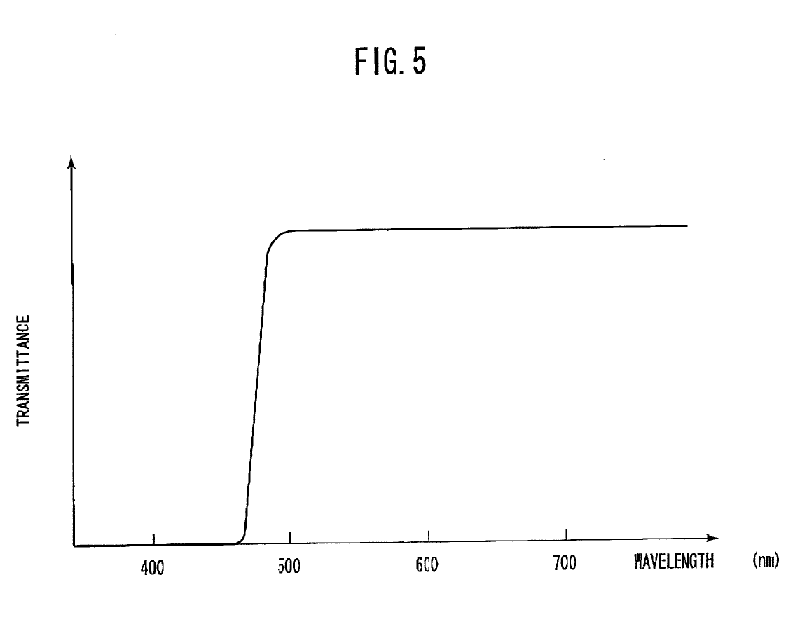

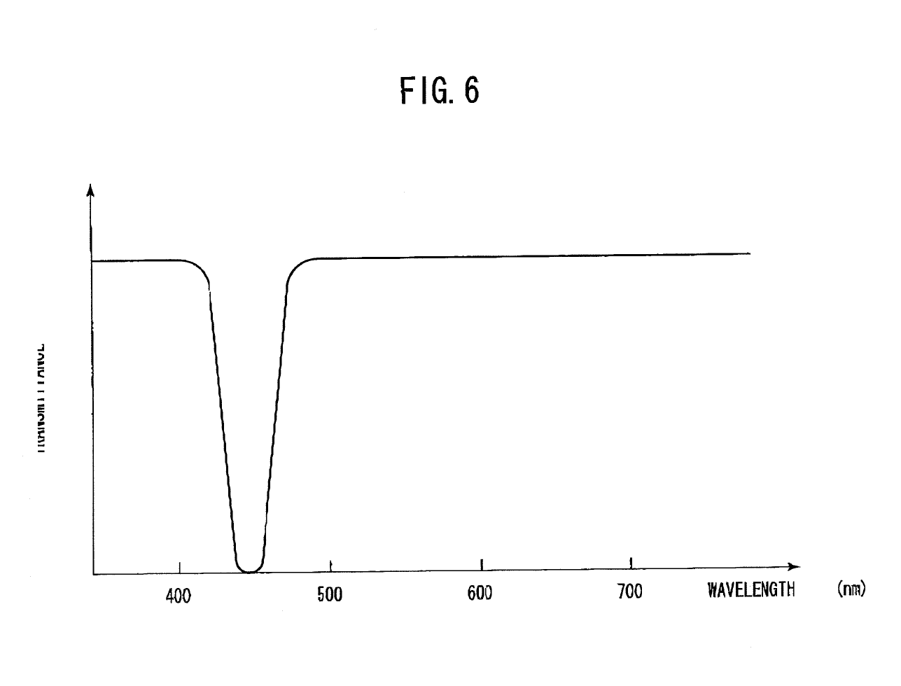

An optical image of the object illuminated by the white light, the first exciting light and/or the second exciting light is captured by the imaging device 52 through the object lens 59 and the exciting-light cut-off filter 55. The properties of the exciting-light cut-off filter 55 differ according to the kind of electronic endoscope 50. For example, a 430 nm cut-off filter which attenuates a light component with a wavelength under 430 nm (see FIG. 4), a 460 nm cut-off filter which attenuates a light component with a wavelength under 460 nm (see FIG. 5), or a 445 nm trap filter which attenuates a light component whose wavelength ranges within a narrow band nearby 445 nm (see FIG. 6) is mounted in the electronic endoscope 50. The exciting-light cut-off filter attenuates a partial light component of the exciting light.

The ROM 56 stores filter information that indicates some properties of the exciting-light cut-off filter 55. When the electronic endoscope 50 is connected to the endoscope processor 20, the filter information is sent to the system controller 21. Based on the received filter information, the system controller determines the observation mode which the autofluorescence endoscope system including the connected electronic endoscope 50 can carry out. The system controller controls the timing controller 22 and the other components according to the determined observation mode. Once the system controller 21 has determined the available observation modes, one of them can be selected by user input operation to the input block 54.

The light component resulting from the property of the exciting-light cut-off filter 55 in an optical image of the subject illuminated by the white light, the first exciting light, and/or the second exciting light is attenuated. An optical image passing through the exciting-light cut-off filter 55 reaches the imaging device 52.

The imaging-device driver 53 drives the imaging device 52 so that the imaging device 52 captures an optical image and generates an image signal according to the captured optical image. The imaging-device driver 53 drives the imaging device 52 based on the clock pulse sent from the timing controller 22. The generated image signal in sent to the image-processing unit 40.

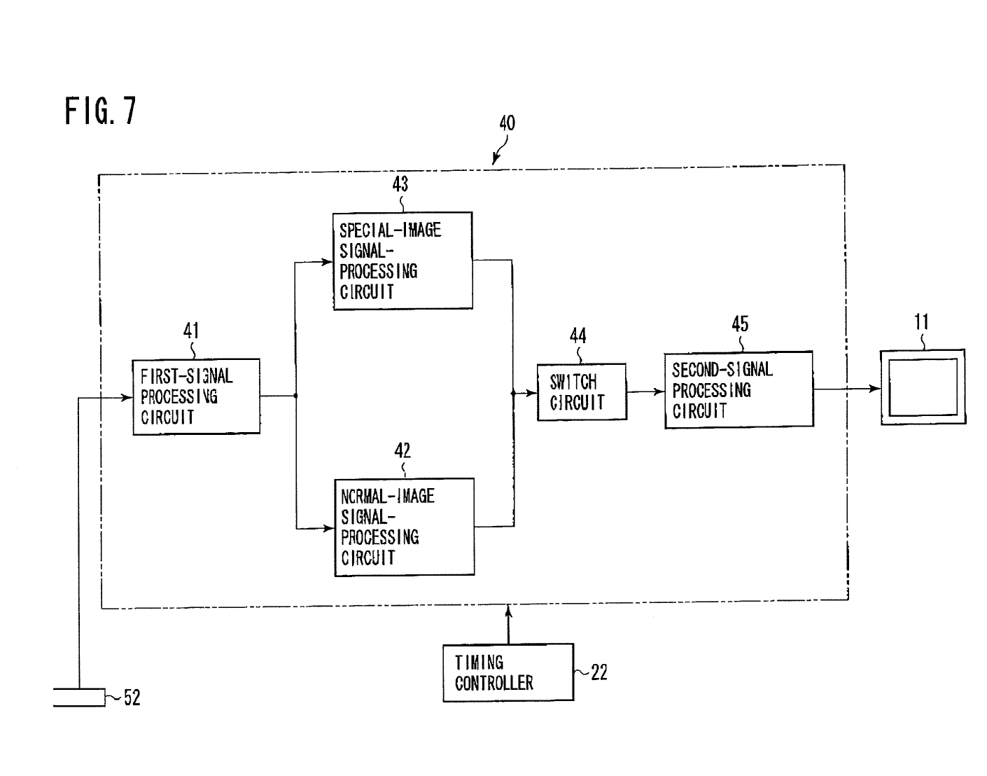

Next, the structure of the image-processing unit 40 is explained using FIG. 7. The image-processing unit 40 comprises a first-signal processing circuit 41, a normal-image signal-processing circuit 42, a special-image signal-processing circuit 43, a switch circuit 44, a second-signal processing circuit, and other components.

The image signal received by the image-processing unit 40 is input to the first-signal processing circuit 41. The first-signal processing circuit 41 digitizes the analog image signal. In addition, the first-signal processing circuit carries out predetermined signal processing, such as color interpolation processing and gamma-correction processing, on the digital image signal.

The image signal, having undergone predetermined signal processing, is sent to the normal-image signal-processing circuit 42 or the special-image signal-processing circuit 43. The normal-image signal-processing circuit 42 carries out predetermined signal processing on an image signal generated when the subject is illuminated by the white light. The special-image signal-processing circuit 43 carries out predetermined signal processing on an image signal generated when the required subject is illuminated by the first and the second exciting lights.

The switch circuit 44 switches between whether the normal-image signal-processing circuit 42 or the special-image signal-processing circuit 43 connect to the second-signal processing circuit 45. The image signal is routed from the circuit selected by the switch circuit 44 to the second-signal processing circuit 45.

As described later, multiple kinds of images can be simultaneously displayed on the monitor 11 in the autofluorescence endoscope system 10. For displaying multiple kinds of images on the monitor 11, whenever the field signal alternates between high and low states, the switch circuit 44 switches between the normal- and special-image signal-processing circuits 42 and 43.

The second-signal processing circuit 45 carries out enlargement processing, scale-down processing, and multi-image display processing as required. In addition, the second-signal processing circuit 45 carries out predetermined signal processing, such as clamp processing and blanking processing on the received image signal or an image signal having undergone required signal processing. Furthermore, the second-signal processing circuit 45 converts the digital image signal to analog form. The analog image signal is sent to the monitor 11, where an image corresponding to the received image signal is displayed.

Next, an observation mode according to a property of the light-source filter 55, some operations of the light-source unit 30 in the observation mode, and a displayed image are explained below.

First, an operation mode, some operations, and the image displayed when the 445 nm trap filter is used are explained as follows.

If an electronic endoscope 50 having the 445 nm trap filter is connected to the endoscope processor 20, the endoscope processor 20 enters one among: normal-image mode, first-autofluorescence image mode, enhanced-image mode, and first-third simultaneous display modes.

When the normal-image mode is selected, the timing controller 22 directs the first and second exciting light sources 38a and 38b to turn off the first and second exciting lights, respectively. In addition, the timing controller 22 directs the first motor 39a to drive the shutter 36 so that it is kept out of the optical path of the white light. The system controller 21 directs the second motor 39b to drive the light-source filter 33 so that the light-source filter 33 is kept out of the optical path of the white light.

Then, the light-source unit 20 continuously emits the white light, which illuminates the required subject. The light reflected off the subject arrives at the object lens 59. The imaging device 52 captures an image where a part of the blue band is attenuated by the exciting-light nut-off filter 55. Although a part of the blue band is attenuated by the exciting-light cut-off filter 55 (the 445 nm trap filter), the remaining blue band components permit visualization as if by unaltered white light.

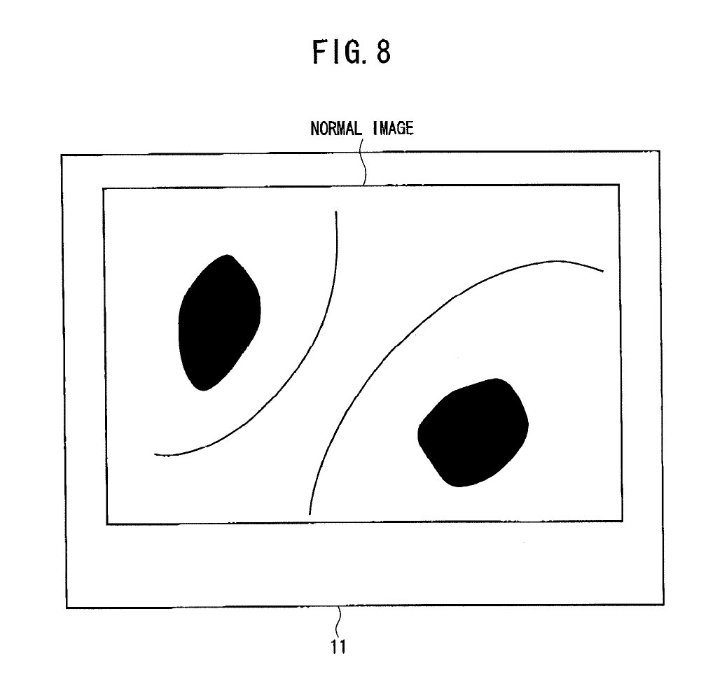

A white-light image signal is generated based on the captured optical image of the subject illuminated by the white light. Based on the white-light image signal, a normal image is displayed on the monitor 11, as shown in FIG. 8.

When the first-autofluorescence image mode is selected, the timing controller 22 turns off the first exciting light source 38a and turns on the second exciting light source 38b. In addition, the timing controller 22 directs the first motor 39a to drive the shutter 36 so that the shutter 36 is held in the optical path of the white light.

Then, the light-source unit 20 continuously emits the second exciting light, which illuminates the subject, which thereupon autofluoresces. Thus, the autofluorescence and reflected light based on the illuminating second exciting light arrives at the object lens 59. The reflected light is attenuated by the exciting-light cut-off filter 55. On the other hand, autofluorescence wavelengths tend to be longer than those of the illuminating exciting light. As a result, autofluorescence is able to pass the exciting-light cut-off filter 55. Accordingly, only the autofluorescence reaches the imaging device 52, which captures an optical image of autofluorescence.

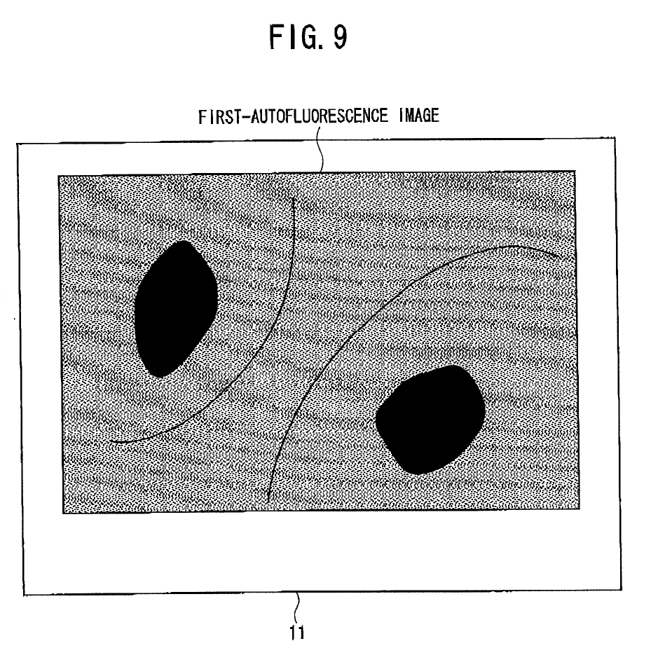

A first-autofluorescence image signal is generated based on the captured optical image of the subject illuminated by the second exciting light. Based on the first-autofluorescence image signal, a first-autofluorescence image is displayed on the monitor 11 as shown in FIG. 9.

When the enhanced-image mode is selected, the timing controller 22 turns on the first exciting light source 38a and turns off the second exciting light source 38b. In addition, the timing controller 22 directs the first motor 39a to drive the shutter 36 so that the shutter 36 is kept out of the optical path of the white light. The system controller 21 directs the second motor 39b to drive the light-source filter 33 so that the light-source filter 33 continues to block the white light.

Then, the light-source unit 20 continuously emits the first exciting light and the green and red light components, which illuminate the subject. The subject illuminated by the first exciting light autofluoresces. Accordingly, the autofluorescence and reflected light based on the illuminated first exciting light and the green and red light components arrives at the object lens 59.

Since the exciting-light cut-off filter 55 is a 445 nm trap filter, the aforementioned light component passes it without attenuation. Accordingly, the autofluorescence and reflected light based on the illuminated first exciting light and the green and red light components reach the imaging device 52, which captures an optical image.

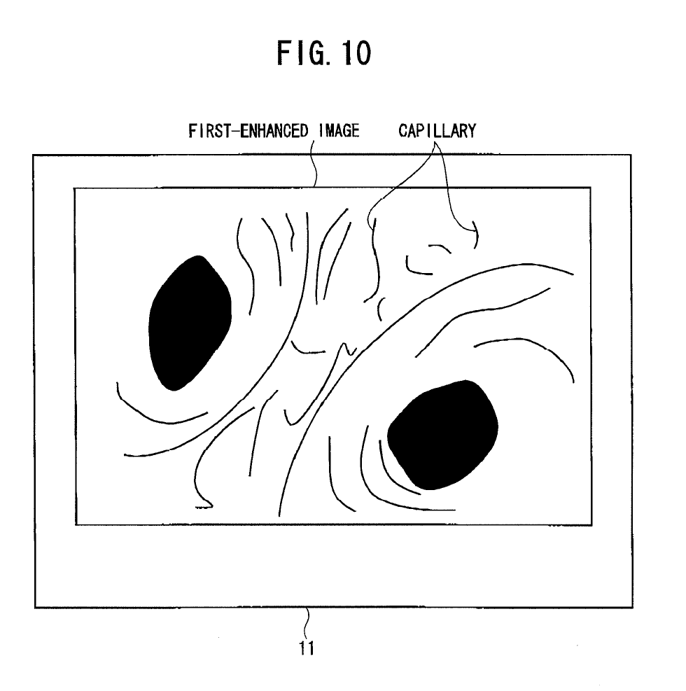

A first-enhanced image signal is generated based on the captured optical image of the subject illuminated by the first exciting light and the green and red light components. Based on the first-enhanced image signal, a first-enhanced image is displayed on the monitor 11, as shown in FIG. 10.

In the first-enhanced image, a full-color image of a capillary is displayed vividly. The wavelength of the first exciting light is short (with a peak at 408 nm) within the blue light band, and the first exciting light is highly absorbed by hemoglobin. Accordingly, by lighting only the first exciting light among the blue light components, optical images of capillaries, which are located in a shallow area under an internal organ wall, can be captured in detail.

In addition, by providing green and red light as well as the first exciting light, a full-color optical image can be captured. Since autofluorescence is dimmer than the reflected light, the autofluorescence component in the entire optical image which reaches the imaging device 52 is indistinguishable.

When the first simultaneous display made is selected, the timing controller 22 directs the first and second exciting light sources 38a and 38b to respectively switch on and off the first and second exciting lights, and the first motor 39a to drive the shutter 36 into and out of the optical path of the white light, whenever the field signal is alternately switched between high and low states. The system controller 21 directs the second motor 39b to drive the light-source filter 33 so that the light-source filter 33 remains in the optical path of the white light.

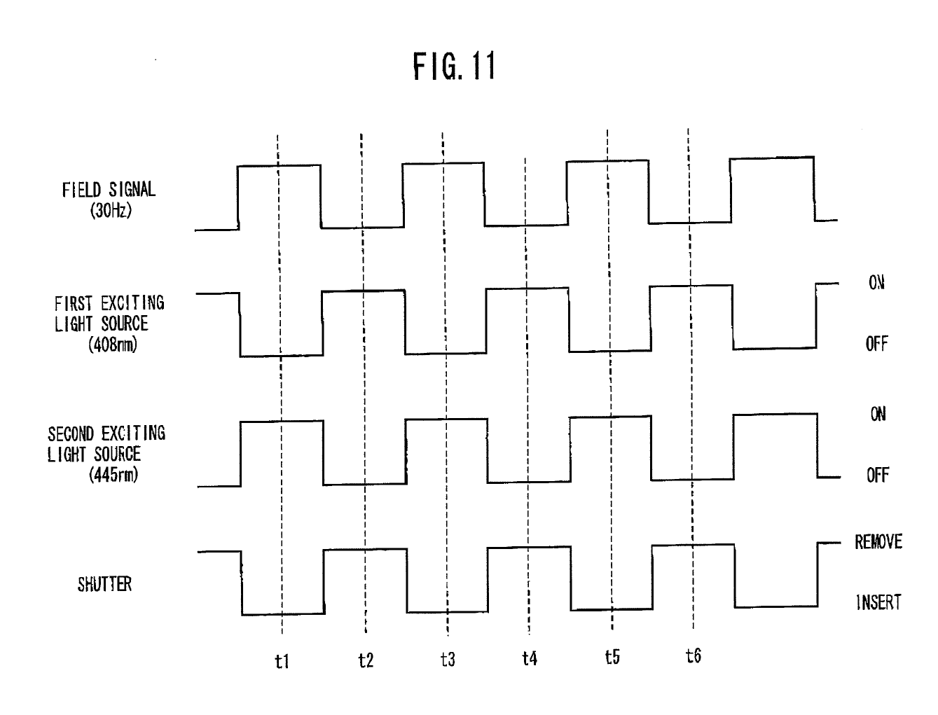

As shown in FIG. 11, at times t1, t3, and t5, the timing controller 22 directs the first exciting light source 38a to turn off, the second exciting light source 38b to light on, and the first motor 39a to drive the shutter into the optical path. Accordingly, the timing controller 22 carries out the same control as the first-autofluorescence image mode at time t1, t3, and t5.

On the other hand, at times t2, t4, and t6, the timing controller 22 directs the first exciting light source 38a to turn on, the second exciting light source 38b to turn off, and the first motor 39a to drive the shutter out of the optical path. Accordingly, the timing controller 22 carries out the same control process as in the enhanced-image mode at time t2, t4, and t6.

The imaging-device controller 53 drives to the imaging device 52 so as to generate one field of an image signal whenever the field signal is alternately switched between high and low states. At times t1, t3, and t5, the first-autofluorescence image signals are generated. At times t2, t4, and t6, the first-enhanced image signals are generated.

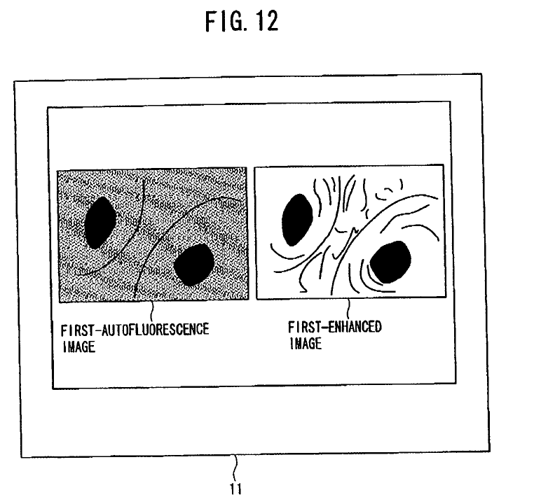

The first-autofluorescence image signals and the first-enhanced image signals are sent to the second-signal processing circuit 45. The second-signal processing circuit 45 carries out multi-image display processing so that the first-autofluorescence image and the first-enhanced image are simultaneously displayed. The video signal, having undergone multi-image display processing, is sent to the monitor, where the first-autofluorescence image and the first-enhanced image are displayed, as shown in FIG. 12.

When the second simultaneous display mode is selected, the timing controller 22 directs the first and second exciting light source 38a and 38b to switch on and off the first and second exciting lights, respectively, and the first motor 39a to drive the shutter 36 into and out of the optical path of the white light, whenever the field signal is alternately switched between high and low states. The system controller 21 directs the second motor 39b to drive the light-source filter 33 so that the light-source filter 33 is kept out of the optical path of the white light.

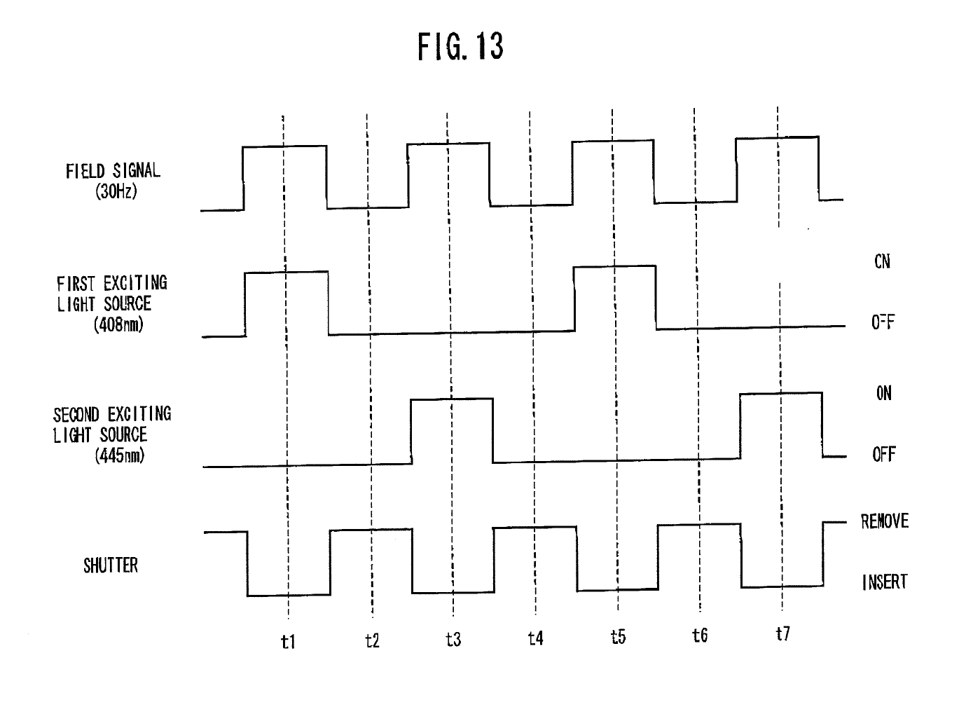

As shown in FIG. 13, at times t1 and t5, the timing controller 22 directs the first exciting light source 38a to turn on, the second exciting light source 38b to turn off, and the first motor 39a to drive the shutter into the optical path. Then, the light-source unit 20 emits the first exciting light at times t1 and t5.

The autofluorescence and reflected light based on the illuminated first exciting light are incident on the object lens 59. Since the first exciting light is a narrow-band light with peak wavelength at 408 nm and the exciting-light out-off filter 55 is a 445 nm trap filter, the optical image formed by the autofluorescence and the reflected light components based on the first exciting light pass the exciting-light cut-off filter 55 without attenuation. Accordingly, both the autofluorescence and the reflected light based on the first exciting light components reach the imaging device 52, which captures an optical image of autofluorescence and reflected light.

As shown in FIG. 13, at times t2, t4, and t6, the timing controller 22 directs the first and second exciting light sources 38a and 38b to turn off and the first motor 39a to drive the shutter 36 out of the optical path. Accordingly, the timing controller 22 carries out the same control as the normal-image mode at time t2, t4, and t6.

As shown in FIG. 13, at times t3 and t7, the timing controller 22 directs the first exciting light source 38a to turn off, the second exciting light source 38b to turn on, and the first motor 39a to drive the shutter 36 into the optical path. Accordingly, the timing controller 22 carries out the same control as the first-autofluorescence image mode at time t3 and t7.

The imaging device controller 53 drives the imaging device 52 so as to generate one field of an image signal whenever the field signal is alternately switched to high and low states. At times t2, t4, and t6, the white-light image signals are generated. At times t3 and t7, the first-autofluorescence image signals are generated.

At times t1 and t5, second-enhanced image signals are generated based on the received optical image by the autofluorescence and the reflected light against the first exciting light.

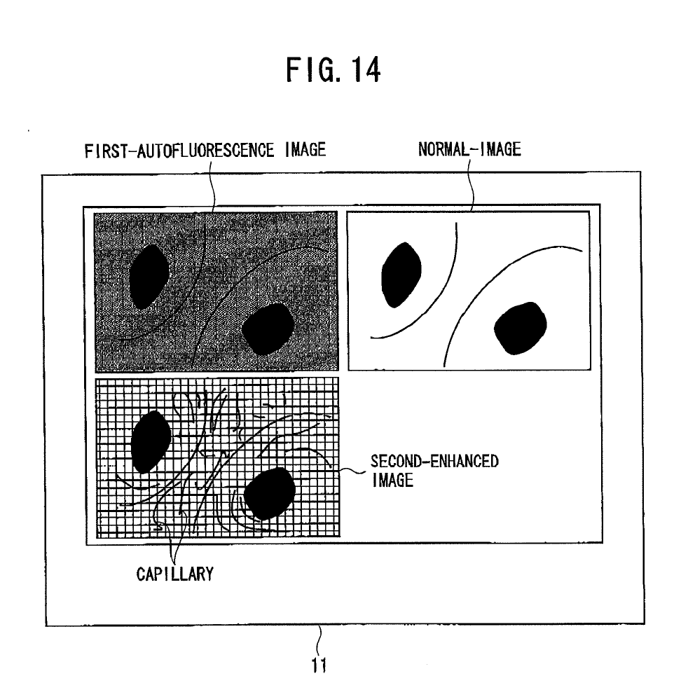

The white-light image signals, the first-autofluorescence image signals, and the second-enhanced image signals are sent to the second-signal processing circuit 45. The second-signal processing circuit 45 carries out multi-image display processing so that the normal image, the first-autofluorescence image and a second-enhanced image are simultaneously displayed. The video signal, having undergone multi-image display processing, is sent to the monitor, where the normal image, the first-autofluorescence image, and the second-enhanced image are displayed, as shown in FIG. 14.

In the second-enhanced image, a monochromatic image of a capillary is displayed vividly. The wavelength of the first exciting light is at the short end of the blue band, and the first exciting light is highly absorbed by hemoglobin. Accordingly, by lighting only the first exciting light among the blue light components, an optical image of a capillary, which is located in a shallow area under an internal organic wall, can be captured in detail. However, it is different from the first-enhanced image, a full-color image cannot be displayed in the second-enhanced image because green and red light components are not illuminated. An autofluorescence component in the entire optical image which reaches the imaging device 52 is indistinguishable, just as in the first-enhanced image.

When the third simultaneous display mode is selected, the timing controller 22 controls the first and second exciting light sources 38a and 38b to switch on and off the first and second exciting light, respectively, and the first motor 39a to drive the shutter 36 in and out of the optical path of the white light, whenever the field signal is alternately switched between high and low states. The system controller 21 controls the second motor 39b to drive the light-source filter 33 so that the light-source filter 33 is kept out of the optical path of the white light.

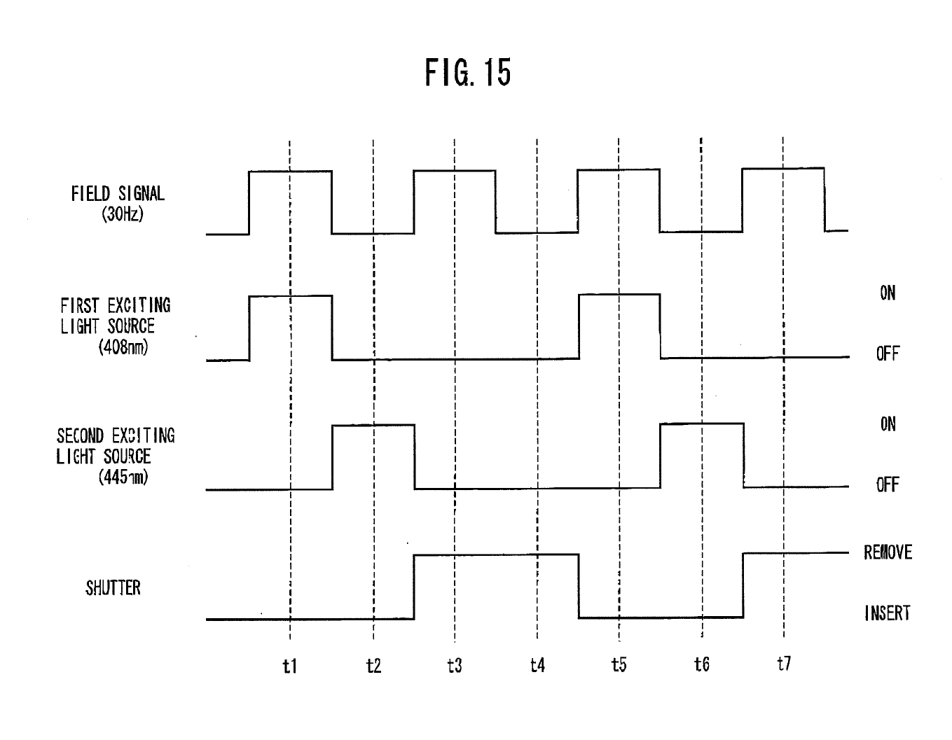

In the third simultaneous display mode, the first and second exciting lights, and the white light are illuminated at different times, as in the second simultaneous display mode. In the second simultaneous display mode, the first exciting light, the white light, the second exciting light, and the white light are repeatedly illuminated in this order. However, in the third simultaneous display mode, the first exciting light, the second exciting light, and the white light are repeatedly illuminated in order. In addition, in contrast to the second simultaneous display mode, the white light is illuminated in two successive fields in the third simultaneous display mode (see FIG. 15).

In the third simultaneous display mode, the normal image, the first-autofluorescence image, and the second-enhanced image are displayed on the monitor 11, as in the second simultaneous display mode. However, the normal image in the third simultaneous display mode is less blurry than one in the second simultaneous display mode because it is based on two successive fields of image signals.

As described above, if the 445 nm trap filter is adapted for the exciting-light cut-off filter 33, a normal image or a subject illuminated by white light, an autofluorescence image from a subject illuminated by exciting light of 445 nm, and an image in which a subject strongly absorbs narrow-band light around 408 nm is vividly displayed can be observed.

Next, an operation mode, some operations, and the image displayed in the case of the 460 nm cut-off filter are explained as follows.

If an electronic endoscope 50 having the 460 nm cut-off filter is connected to the endoscope processor 20, the endoscope processor 20 enters an operation mode from among the normal-image mode, the second-fifth autofluorescence image modes, and the fourth-seventh simultaneous display modes.

In the normal-image mode, the operations of the components of the light-source unit 30 and the image displayed on the monitor 11 are the same as in the electronic endoscope with the 445 nm trap filter.

When the second-autofluorescence image mode is selected, the timing controller 22 directs the first and second exciting light sources 38a and 38b to turn on the first and second exciting lights and the first motor 39a to drive the shutter 36 so as to keep in the optical path of the white light.

Then, the light-source unit 20 continuously emits the first and second exciting lights, which illuminate the subject. When illuminated by the first and second exciting lights, the subject autofluoresces. The reflected light is attenuated by the exciting-light cut-off filter 55. Accordingly, only the autofluorescence reaches the imaging device 52, which captures an optical image of the autofluorescence based on the first and second exciting lights.

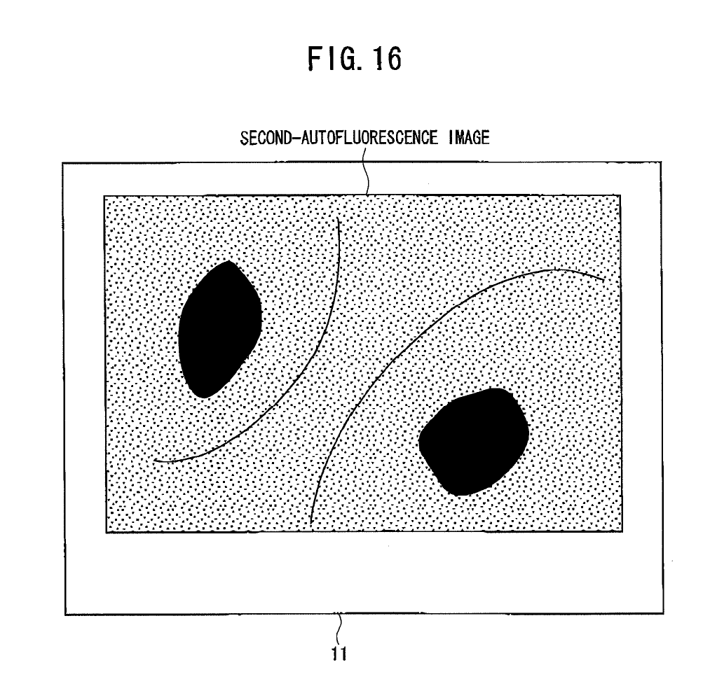

A second-autofluorescence image signal is generated based on the received optical image of the subject illuminated by the first and second exciting lights. Based on the second-autofluorescence image signal, a second-autofluorescence image is displayed on the monitor 11, as shown in FIG. 16. It is different from the first-autofluorescence image in that an image of the autofluorescence based on the first exciting light is also displayed in the second-autofluorescence image.

When the third-autofluorescence image mode is selected the timing controller 22 directs the first exciting light source 38a to turn on and the second exciting light source 38b to turn off. In addition, the timing controller 22 directs the first motor 39a to drive the shutter 36 so that the shutter 36 is kept in the optical path of the white light.

Then, the light-source unit 20 continuously emits the first exciting light, which illuminates the required subject. The reflected light is attenuated by the exciting-light cut-off filter 55. Accordingly, only the autofluorescence reaches the imaging device 52, which captures an optical image of the autofluorescence based on the first exciting light.

A third-autofluorescence image signal is generated based on the captured optical image of the subject illuminated by the first exciting light. Based on the third-autofluorescence image signal, a third-autofluorescence image is displayed on the monitor 11. It is different from the first-autofluorescence image in that the third-autofluorescence image is an optical image of autofluorescence based on the first exciting light.

When the fourth-autofluorescence image mode is selected, the timing controller 22 directs the first exciting light source 38a to turn off and the second exciting light sources 38b to turn on. In addition, the timing controller 22 directs the first motor 39a to drive the shutter 36 so that the shutter 36 is kept in the optical path of the white light.

In the fourth-autofluorescence image mode, the optical image of autofluorescence based on the second exciting light is displayed, as in the first-autofluorescence image mode.

When the fifth-autofluorescence image node is selected, the timing controller 22 directs the first and second exciting light sources 38a and 38b to switch on and off, whenever the field signal is alternately switched between high and low states. On the other hand, the timing controller 22 directs the first motor 39a to drive the shutter 36 so as to keep it inserted in the optical path of the white light.

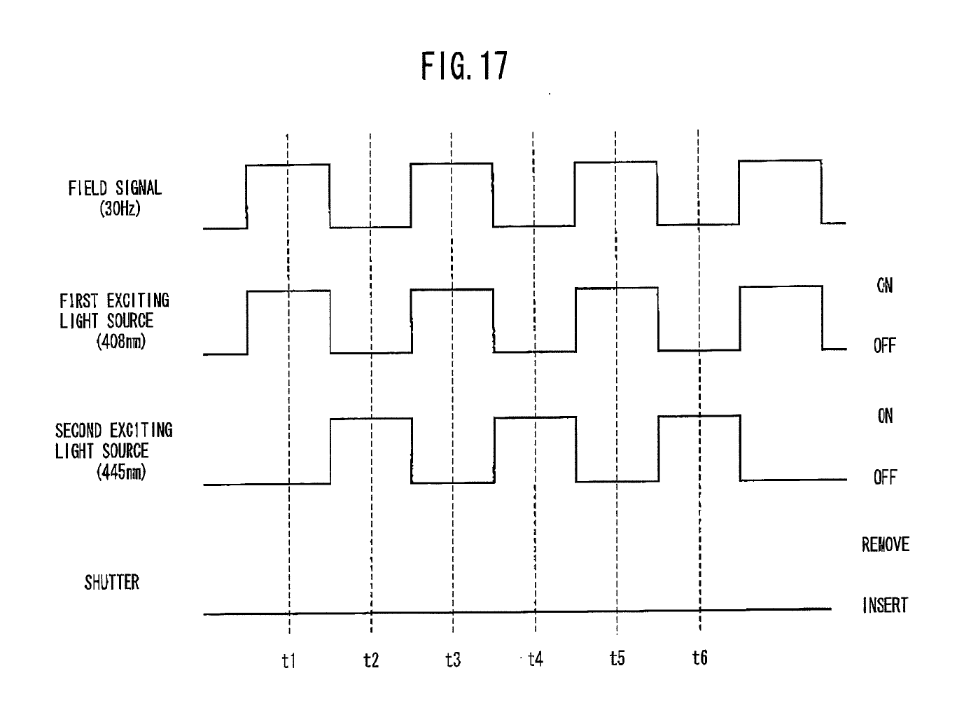

As shown in FIG. 17, at times t1, t3, and t5, the timing controller 22 directs the first exciting light source 38a to turn on, and the second exciting light source 38b to turn off. Accordingly, the timing controller 22 carries out the same control as the third-autofluorescence image mode at times t1, t3, and t5.

On the other hand, at times t2, t4, and t6, the timing controller 22 directs the first exciting light source 38a to turn off, the second exciting light source 38b to turn on. Accordingly, the timing controller 22 carries out the same control as the fourth-autofluorescence image mode at time t2, t4, and t6.

The imaging-device controller 53 drives the imaging device 52 so as to generate one field of an image signal whenever the field signal is alternately switched between high and low states. At times t1, t3, and t5, the third-autofluorescence image signals are generated. At times t2, t4, and t6, the fourth-autofluorescence image signals are generated.

The special-image signal-processing circuit 43 generates a fifth-autofluorescence image signal corresponding to a fifth-autofluorescence image based on the third- and fourth-autofluorescence image signals. In the fifth-autofluorescence image, edges displayed in the third-autofluorescence image but not in the fourth-autofluorescence image, edges not displayed in the third-autofluorescence image but in the fourth-autofluorescence image, edges displayed both in the third- and fourth-autofluorescence images, or edges displayed either only in the third- or fourth-autofluorescence images, are extracted.

The fifth-autofluorescence image signal is sent to the monitor 11 via the second-signal processing circuit 45. The fifth-autofluorescence image is displayed on the monitor 11.

In the fourth simultaneous display mode, operations of the light-source unit 30 alternate between those of the normal-image mode and those of the second-autofluorescence image mode whenever the field signal alternates between high and low states. Accordingly, in the fourth simultaneous display mode, the normal image and the second-autofluorescence image are simultaneously displayed on the monitor 11.

In the fifth simultaneous display mode, operations of the light-source unit 30 alternate between those of the normal-image mode and those of the third-autofluorescence image mode whenever the field signal alternates between high and low states. Accordingly, in the fifth simultaneous display mode, the normal image and the third-autofluorescence image are simultaneously displayed on the monitor 11.

In the sixth simultaneous display mode, operations of the light-source unit 30 alternate between those of the normal-image mode and those of the fourth-autofluorescence image mode whenever the field signal alternates between high and low states. Accordingly, in the sixth simultaneous display mode, the normal image and the fourth-autofluorescence image are simultaneously displayed on the monitor 11.

In the seventh simultaneous display mode, operations of the light-source unit 30 alternate between those of the normal-image mode and those of the fifth-autofluorescence image mode whenever the field signal alternates between high and low states. Accordingly, in the seventh simultaneous display mode, the normal image and the fifth-autofluorescence image are simultaneously displayed on the monitor 11. In addition, in the seventh simultaneous display mode, a plurality of images from among the normal image, the third-, fourth-, and fifth-autofluorescence images can be simultaneously displayed because the third- and fourth-autofluorescence image signals are generated.

As described above, if the 460 nm cut-off filter is adapted for the exciting-light cut-off filter 33, the normal image, an autofluorescence image from a required subject illuminated by exciting light of 408 nm and 445 nm, an autofluorescence image from a required subject illuminated by exciting light either of 408 nm or 445 nm, and an image synthesized based on autofluorescence images against the exciting light of 408 nm n and 445 nm can be observed.

Next, an operation mode, some operations, and the image displayed in the case of the 430 nm cut-off filter are explained as follows.

If an electronic endoscope 50 having the 430 nm cut-off filter is connected to the endoscope processor 20, the endoscope processor 20 carries out one from among the normal-image mode, the sixth-autofluorescence image mode, and the eighth simultaneous display mode.

In the normal-image mode, operations of each component of the light-source unit 30 and an image displayed on the monitor 11 are same au the electronic endoscope with the 445 nm trap filter.

When the sixth-autofluorescence image mode is selected, the timing controller 22 controls the first and second exciting light sources 38a and 38b and the first motor 39a, just as in the third-autofluorescence image mode. Accordingly, the light-source unit 20 continuously emits the first exciting light, which illuminates the subject. The reflected light of the first exciting light is attenuated by the exciting-light cut-off filter 55, and only the autofluorescence reaches the imaging device 52, which captures an optical image of autofluorescence based on the first exciting light.

A sixth-autofluorescence image signal is generated based on the captured optical image of the subject illuminated by the first exciting light. Based on the sixth-autofluorescence image signal, a sixth-autofluorescence image is displayed on the monitor 11. An optical image of the autofluorescence component whose wavelength band is under 460 nm in response to the first exciting light is not included in the third-autofluorescence image. On the other hand, an optical image of the autofluorescence component whose wavelength band ranges between 430 nm and 460 nm is included in the sixth-autofluorescence image.

In the eighth simultaneous display mode operations of the light-source unit 30 alternate between those of the normal-image mode and those of the sixth-autofluorescence image mode whenever the field signal alternates between high and low states. Accordingly, in the eighth simultaneous display mode, the normal image and the sixth-autofluorescence image are simultaneously displayed on the monitor 11.

As described above, if the 430 nm cut-off filter is adapted for the exciting-light cut-off filter 33, the normal image and an autofluorescence image from a subject illuminated by exciting light of 408 nm can be observed.

In addition, when an electronic endoscope without a filter which attenuates exciting light is connected to the endoscope processor 20, the endoscope processor can carry out one from among the normal-image mode, the enhanced-image mode, and a ninth simultaneous display mode, where the normal image and the first-enhanced image are simultaneously displayed.

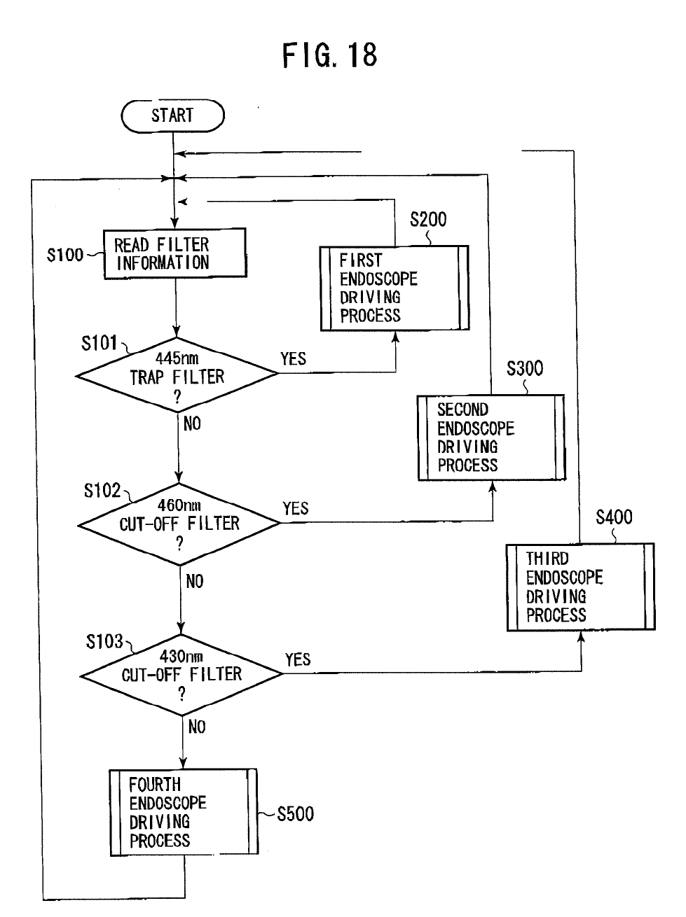

Next, operations of the components of the light-source unit 30 and the electronic endoscope 50 carried out by the electronic endoscope 20 are explained below using the flowcharts of FIGS. 18-34.

The control processes of the light-source unit 30 and the electronic endoscope 50, and the image signal processes of this embodiment start when the electronic endoscope 50 is connected to the endoscope processor 20. In addition, the control processes and the image signal processes finish when the power of the endoscope processor 20 is switched off.

As shown in FIG. 18, at step S100, the filter information is read from the ROM 56 of the electronic endoscope 50. In the following steps S101-S103, a property of the exciting-light cut-off filter 55 mounted in the connected electronic endoscope 50 is specified based on the filter information. The exact processes at step S101-S103 are explained below.

At step S101, it is determined whether the exciting-light cut-off filter 55 is a 445 nm trap filter. If the exciting-light cut-off filter 55 is not a 445 nm trap filter, the process proceeds to step S102. On the other hand, if the exciting-light cut-off filter 55 is a 445 nm trap filter, the process proceeds to the subroutine for the first endoscope driving process (S200). Some operations in the subroutine for the first endoscope driving process are explained later. After completing the first endoscope driving process, the process returns to step S100.

At step S102, it is determined whether the exciting-light cut-off filter 55 is a 460 nm cut-off filter. If the exciting-light cut-off filter 55 is not a 460 nm cut-off filter, the process proceeds to step S103. On the other hand, if the exciting-light cut-off filter 55 is a 460 nm cut-off filter, the process proceeds to the subroutine for a second endoscope driving process (S300). Some operations in the second endoscope driving process are explained later. After completing the second endoscope driving process, the process returns to step S100.

At step S102, it is determined whether the exciting-light cut-off filter 55 is a 430 nm cut-off filter. If the exciting-light out-off filter 55 is a 430 nm cut-off filter, the process proceeds to the subroutine for a third endoscope driving process (S400). Some operations in the third endoscope driving process are explained later. After completing the third endoscope driving process, the process returns to step S100.

On the other hand, if the exciting-light cut-off filter 55 is not a 430 nm cut-off filter, the process proceeds to the subroutine for a fourth endoscope driving process (S500). Some operations in the fourth endoscope driving process are explained later. After completing the fourth endoscope driving process, the process returns to step S100.

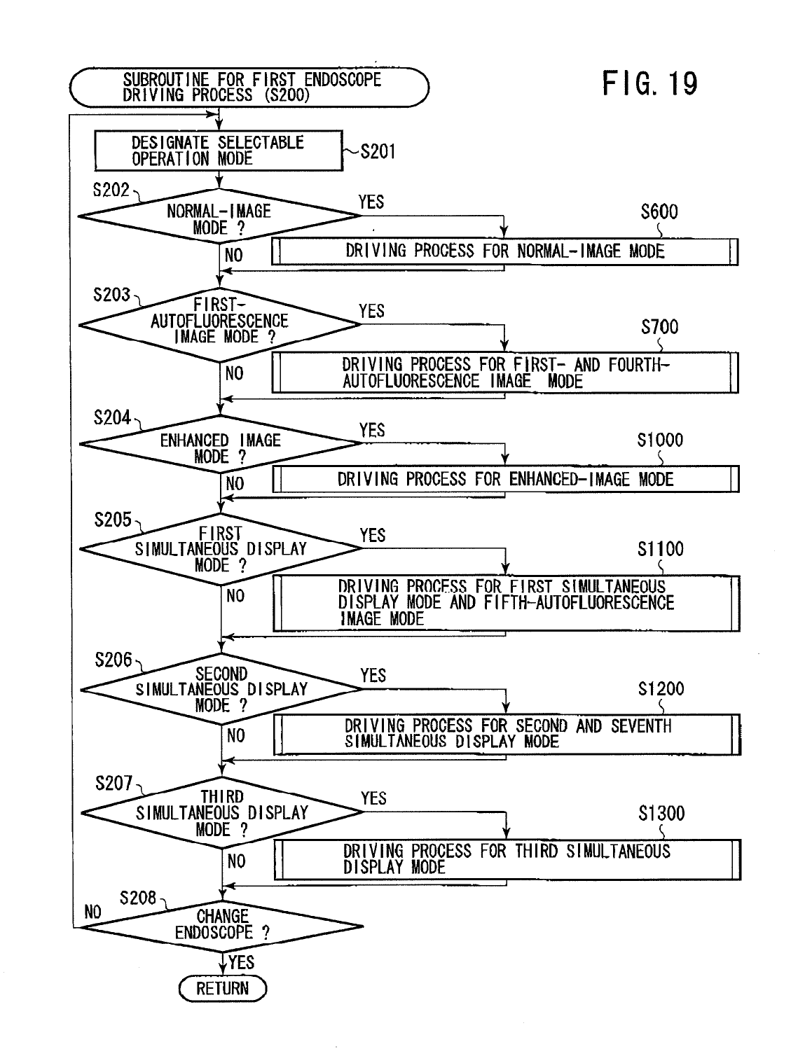

Next, a subroutine for the first endoscope driving process (S200) is explained using the flowchart of FIG. 19 At step S201, the observation modes which can be carried cut when the exciting-light out-off filter 55 is a 445 nm trap filter are made selectable.

At step S202, it is determined whether an input for selection of the normal-image mode is detected. If the normal-image mode is selected, the process proceeds to the subroutine for the driving process for the normal-image mode (S600). Some operations in the subroutine for the driving process for the normal-image mode are explained later. After completing the driving process for the normal-image mode or if the normal-image mode is not selected, the process proceeds to step S203.

At step S203, it is determined whether an input for selection of the first-autofluorescence image mode is detected. If the first-autofluorescence image mode is selected, the process proceeds to the subroutine for a driving process for the first- and fourth-autofluorescence image modes (S700). Some operations in the subroutine for the driving process for the first- and fourth-autofluorescence image modes are explained later. After completing the driving process for the first- and fourth-autofluorescence image modes or if the first-autofluorescence image mode not being selected, the process proceeds to step S204.

At step S204, it is determined whether an input for selection of the enhanced-image mode is detected. If the enhanced-image mode is selected, the process proceeds to the subroutine for the driving process for the enhanced-image mode (S1000). Some operations in the subroutine for the driving process for the enhanced-image mode are explained later. After completing the driving process for the enhanced-image mode or if the enhanced-image mode is not selected, the process proceeds to step S205.

At step S205, it is determined whether an input for selection of the first simultaneous display mode is detected. If the first simultaneous display mode is selected, the process proceeds to the subroutine for the driving process for the first simultaneous display mode and the fifth-autofluorescence image mode (S1100). Some operations in the subroutine for the driving process for the first simultaneous display mode and the fifth-autofluorescence image mode are explained later. After completing the driving process for the first simultaneous display mode and the fifth-autofluorescence image mode, or if the first simultaneous display mode is not selected, the process proceeds to step S206.

At step S206, it is determined whether an input for selection of the second simultaneous display mode is detected. If the second simultaneous display mode is selected, the process proceeds to the subroutine for a driving process for the second and seventh simultaneous display modes (S1200). Some operations in the subroutine for the driving process for the second and seventh simultaneous display modes are explained later. After completing the driving process for the second and seventh simultaneous display modes or if the second simultaneous display mode is not selected, the process proceeds to step S207.

At step S207, it is determined whether an input for selection of the third simultaneous display mode is detected. If the third simultaneous display mode is selected, the process proceeds to the subroutine for the driving process for the third simultaneous display mode (S1300). Some operations in the subroutine for the driving process for the third simultaneous display mode are explained later. After completing the driving process for the third simultaneous display mode or if the third simultaneous display mode is not selected, the process proceeds to step S208.

At step S208, it is determined whether the electronic endoscope 50 connected to the endoscope processor 20 has changed. If the electronic endoscope 50 has not changed, the process returns to step S201 and steps S201-S208 are repeated. If the electronic endoscope 50 has changed, the process returns to step S100.

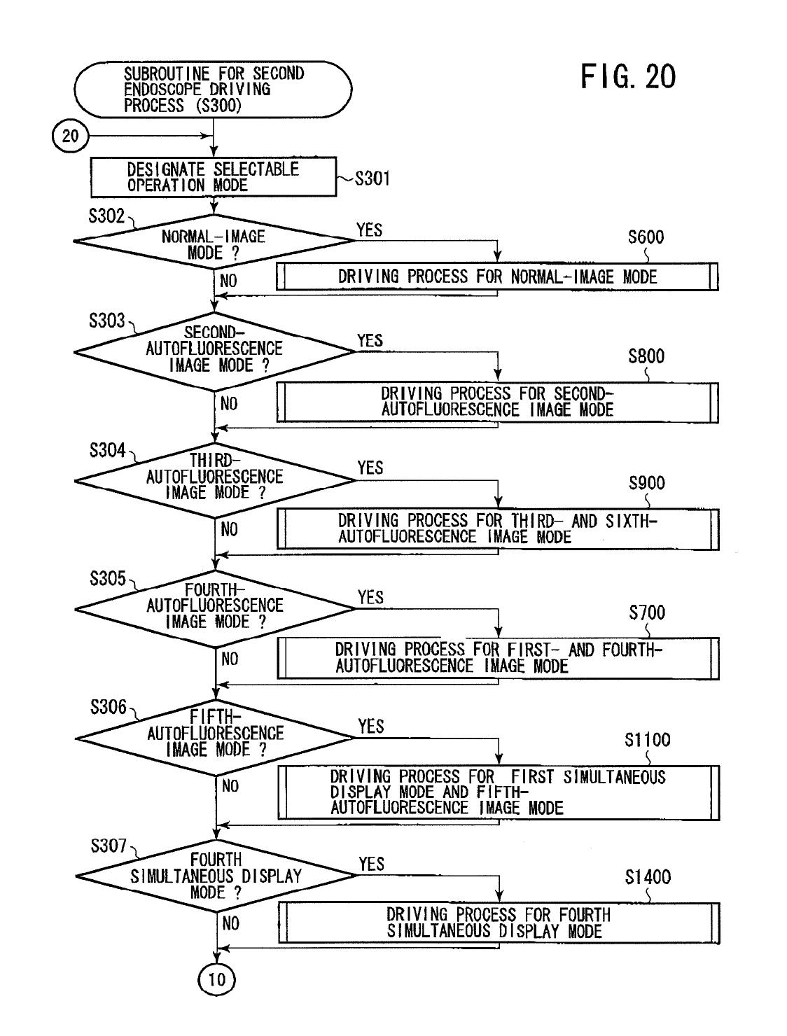

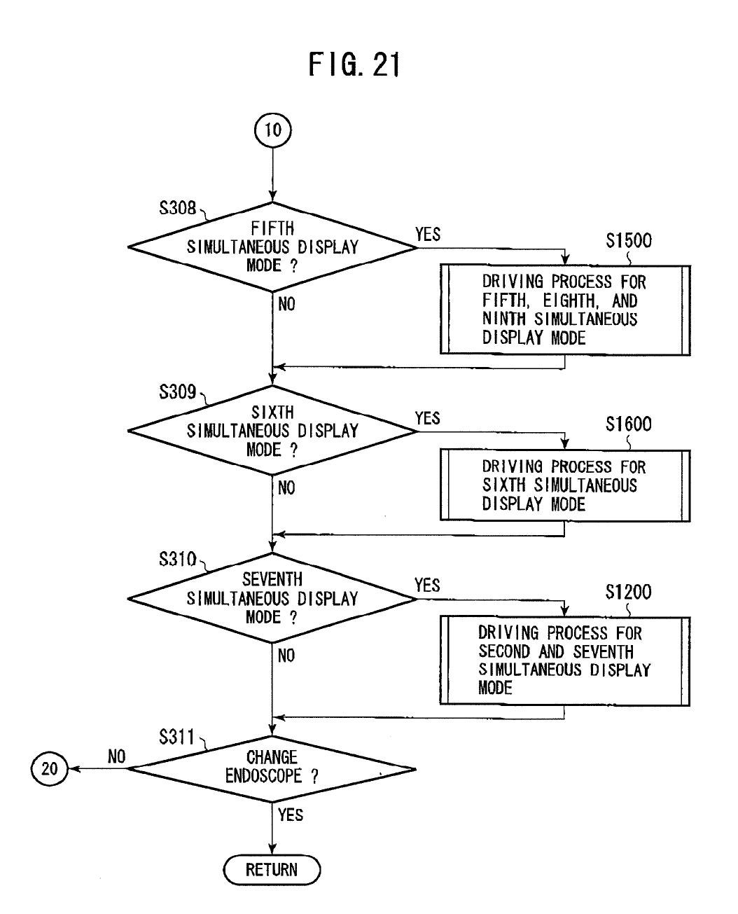

Next, a subroutine for the second endoscope driving process (S300) is explained using the flowchart of FIGS. 20 and 21. At step S301, the observation modes which can be carried out when the exciting-light cut-off filter 55 is 460 nm cut-off filter are made selectable.

At step S302, it is determined whether an input for selection of the normal-image mode is detected. If the normal-image mode is selected, the process proceeds to the subroutine for the driving process for the normal-image mode (S600). Some operations in the subroutine for the driving process for the normal-image mode are explained later. After completing the driving process for the normal-image mode or if the normal-image mode is not selected, the process proceeds to step S303.

At step S303, it is determined whether an input for selection or the second-autofluorescence image mode is detected. If the second-autofluorescence image mode is selected, the process proceeds to the subroutine for a driving process for the second-autofluorescence image made (S800). Some operations in the subroutine for the driving process for the second-autofluorescence image mode are explained later. After completing the driving process for the second-autofluorescence image mode or if the second-autofluorescence image mode is not selected, the process proceeds to step S304.

At step S304, it is determined whether an input for selection of the third-autofluorescence image mode is detected. If the third-autofluorescence image mode is selected, the process proceeds to the subroutine for the driving process for the third- and sixth-autofluorescence image modes (S900). Some operations in the subroutine for the driving process for the third- and sixth-autofluorescence image inode are explained later. After completing the driving process for the third- and sixth-autofluorescence image modes or if the third-autofluorescence image mode is not selected, the process proceeds to step S305.

At step S305, it is determined whether an input for selection of the fourth-autofluorescence image mode is detected. If the fourth-autofluorescence image mode is selected, the process proceeds to the subroutine for the driving process for the first- and fourth-autofluorescence image modes (S700). Some operations in the subroutine for the driving process for the first- and fourth-autofluorescence image modes are explained later. After completing the driving process for the first- and fourth-autofluorescence image modes or if the fourth-autofluorescence image mode is not selected, the process proceeds to step S306.

At step S306, it is determined whether an input for selection of the fifth-autofluorescence image mode is detected. If the fifth-autofluorescence image mode is selected, the process proceeds to the subroutine for a driving process for the first simultaneous display mode and the fifth-autofluorescence image mode (S1100). Some operations in the subroutine for the driving process for the first simultaneous display mode and the fifth-autofluorescence image mode are explained later. After completing the driving process for the first simultaneous display mode and the fifth-autofluorescence image mode or if the fifth-autofluorescence image mode is not selected, the process proceeds to step S307.

At step S307, it is determined whether an input for selection of the fourth simultaneous display mode is detected. If the fourth simultaneous display mode is selected, the process proceeds to the subroutine for the driving process for the fourth simultaneous display mode (S1400). Some operations in the subroutine for the driving process for the fourth simultaneous display mode are explained later. After completing the driving process for the fourth simultaneous display mode or if the fourth simultaneous display mode is not selected, the process proceeds to step S308.

At step S308, it is determined whether an input for selection of the fifth simultaneous display mode is detected. If the fifth simultaneous display mode is selected, the process proceeds to the subroutine for the driving process for the fifth, eighth, and ninth simultaneous display modes (S1500). Some operations in the subroutine for the driving process for the fifth, eighth, and ninth simultaneous display modes are explained later. After completing the driving process for the fifth, eighth, and ninth simultaneous display modes or if the fifth simultaneous display mode is not selected, the process proceeds to step S309.

At step S309, it is determined whether an input for selection of the sixth simultaneous display mode is detected. If the sixth simultaneous display mode is selected, the process proceeds to the subroutine for the driving process for the sixth simultaneous display mode (S1600). Some operations in the subroutine for the driving process for the sixth simultaneous display mode are explained later. After completing the driving process for the sixth simultaneous display mode or if the sixth simultaneous display mode is not selected, the process proceeds to step S310.

At step S310, it is determined whether an input for selection of the seventh simultaneous display mode is detected. If the seventh simultaneous display mode is selected, the process proceeds to the subroutine for the driving process for the second and seventh simultaneous display modes (S1200). Some operations in the subroutine for the driving process for the second and seventh simultaneous display modes are explained later. After completing the driving process for the second and seventh simultaneous display modes or if the seventh simultaneous display mode is not selected, the process proceeds to step S311.

At step S311, it is determined whether the electronic endoscope 50 connected to the endoscope processor 20 has changed. If the electronic endoscope 50 has not changed, the process returns to step S301 and steps S301-S311 are repeated. If the electronic endoscope 50 has changed, the process returns to step S100.

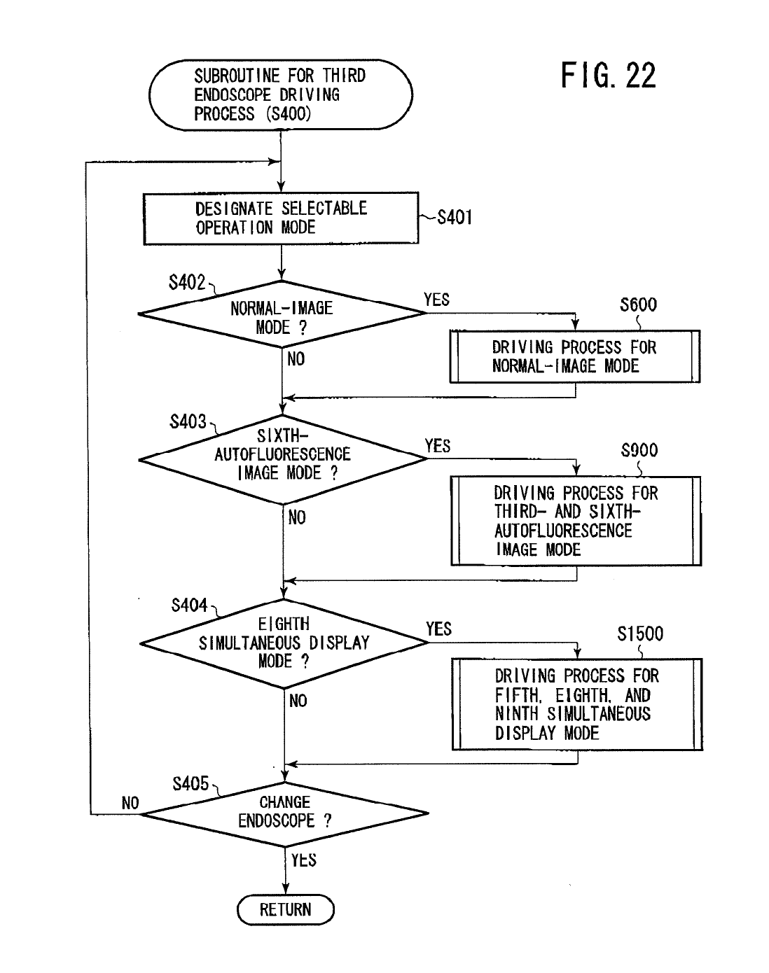

Next, a subroutine for the third endoscope driving process (S400) is explained using the flowchart of FIG. 22. At step S401, the observation modes which can be carried out when the exciting-light out-off filter 55 is 430 nm cut-off filter are made selectable.

At step S402, it is determined whether an input for selection of the normal-image mode is detected. If the normal-image mode is selected, the process proceeds to the subroutine for the driving process for the normal-image mode (S600). Some operations in the subroutine for the driving process for the normal-image mode are explained later. After completing the driving process for the normal-image mode or if the normal-image mode is not selected, the process proceeds to step S403.

At step S403, it is determined whether an input for selection of the sixth-autofluorescence image mode is detected. If the sixth-autofluorescence image mode is selected, the process proceeds to the subroutine for a driving process for the third- and sixth-autofluorescence image modes (S900). Some operations in the subroutine for the driving process for the third- and sixth-autofluorescence image modes are explained later. After completing the driving process for the third- and sixth-autofluorescence image modes or if the sixth-autofluorescence image mode is not selected, the process proceeds to step S404.

At step S404, it is determined whether an input for selection of the eighth simultaneous display mode is detected. If the eighth simultaneous display mode is selected, the process proceeds to the subroutine for the driving process for the fifth, eighth, and ninth simultaneous display modes (S1500). Some operations in the subroutine for the driving process for the fifth, eighth, and ninth simultaneous display modes are explained later. After completing the driving process for the fifth, eighth, and ninth simultaneous display modes or if the eighth simultaneous display mode is not selected, the process proceeds to step S405.

At step S405, it is determined whether the electronic endoscope 50 connected to the endoscope processor 20 has changed. If the electronic endoscope 50 has not changed, the process returns to step S401 and steps S401-S405 are repeated. If the electronic endoscope 50 has changed, the process returns to step S100.

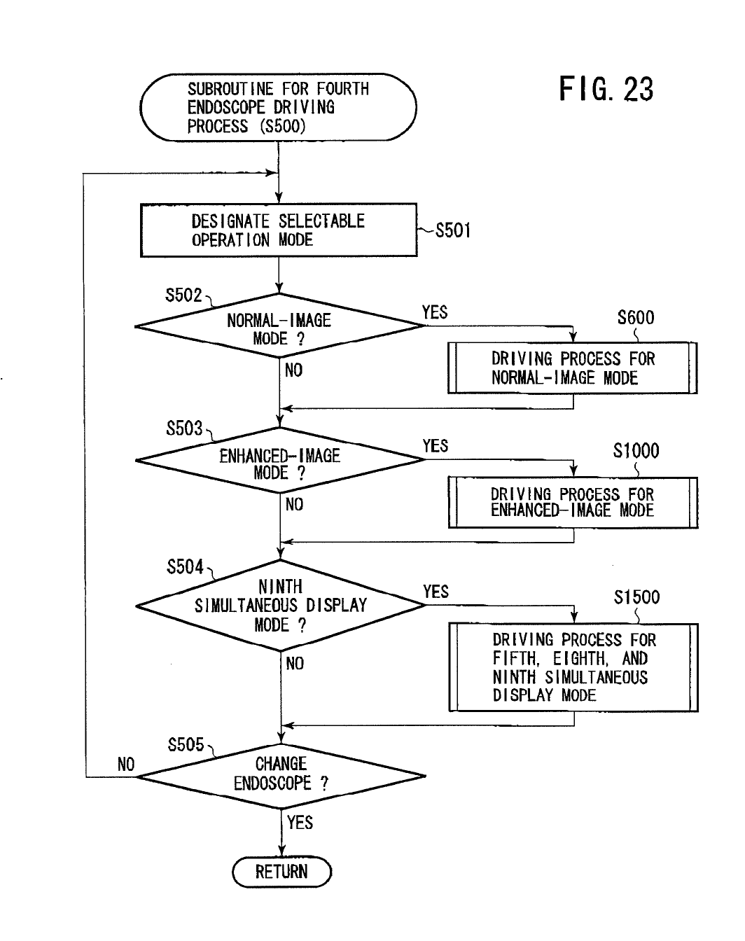

Next, a subroutine for the fourth endoscope driving process (S500) is explained using the flowchart of FIG. 23. At step S501, the observation modes which can be carried out when the exciting-light cut-off filter 55 is not mounted in front of the receiving surface of the imaging device 52 are made selectable.

At step S502, it is determined whether an input for selection of the normal-image mode is detected. If the normal-image mode is selected, the process proceeds to the subroutine for the driving process for the normal-image mode (S600). Some operations in the subroutine for the driving process for the normal-image mode are explained later. After completing the driving process for the normal-image mode or if the normal-image mode is not selected, the process proceeds to step S503.

At step S503, it is determined whether an input for selection of the enhanced-image mode is detected. If the enhanced-image mode is selected, the process proceeds to the subroutine for the driving process for the enhanced-image mode (S1000). Some operations in the subroutine for the driving process for the enhanced-image mode are explained later. After completing the driving process for the enhanced-image mode or if the enhanced-image mode is not selected, the process proceeds to step S504.

At step S504, it is determined whether an input for selection of the ninth simultaneous display mode is detected. If the ninth simultaneous display mode is selected, the process proceeds to the subroutine for the driving process for the fifth, eighth, and ninth simultaneous display modes (S1500). Some operations in the subroutine for the driving process for the fifth, eighth, and ninth simultaneous display modes are explained later. After completing the driving process for the fifth, eighth, and ninth simultaneous display modes or if the ninth simultaneous display mode is not selected, the process proceeds to step S505.

At step S505, it is determined whether the electronic endoscope 50 connected to the endoscope processor 20 has changed. If the electronic endoscope 50 has not changed, the process returns to step S501 and steps S501-S505 are repeated. If the electronic endoscope 50 has changed, the process returns to step S100.

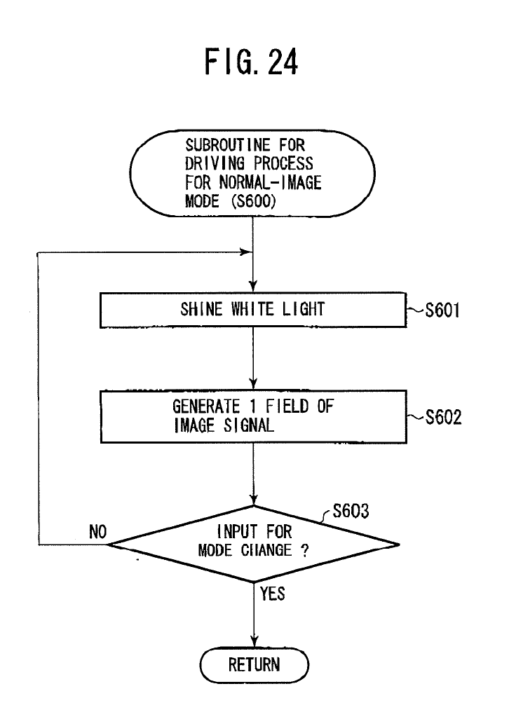

Next, a subroutine for the driving process for the normal-image mode (S600) is explained using the flowchart of FIG. 24. At step S601, by turning on the first and second exciting light sources 38a and 38b and removing the shutter 36 from the optical path of the reference-light source 31, a subject is illuminated by white light.

At step S602, one field of an image signal is ordered to be generated. After generating an image signal, the process proceeds to step S603, where it is determined whether an input for changing the observation mode is detected. If there is no input for changing, the process returns to step S601. Until the input for changing is detected, steps S601-S603 are repeated. If there is input for changing, the process returns to a former subroutine for the endoscope driving process.

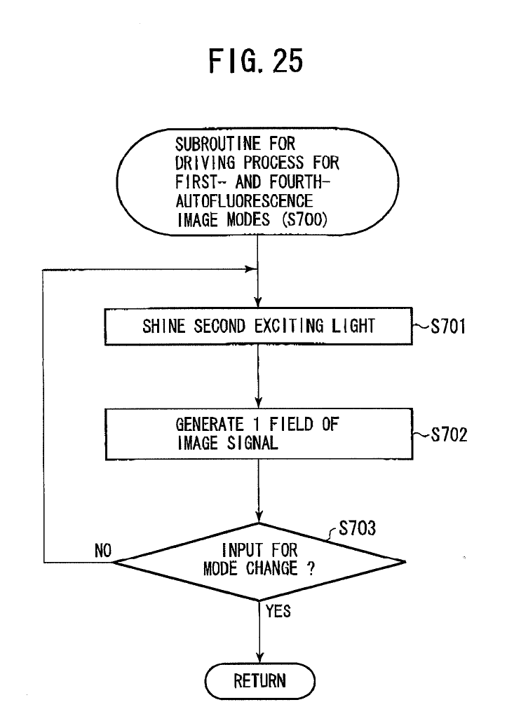

Next, a subroutine for the driving process for the first- and fourth-autofluorescence image modes (S700) is explained using the flowchart of FIG. 25. At step S701, by turning off the first exciting light source 38a, turning on the second exciting light source 38b, and inserting the shutter 36 into the optical path of the reference-light source 31, a subject is illuminated by the second exciting light.

At step S702, one field of an image signal is ordered to be generated. After generating an image signal, the process proceeds to step S703, where it is determined whether an input for changing the observation mode is detected. If there is no input for changing, the process returns to step S701. Until the input for changing is detected, steps S701-S703 are repeated. If there is input for changing, the process returns to a former subroutine for the endoscope driving process.

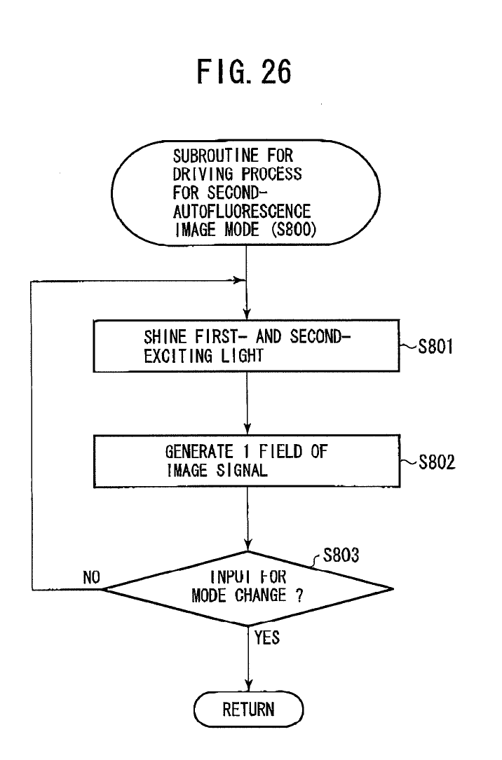

Next, a subroutine for the driving process for the second-autofluorescence image mode (S800) is explained using the flowchart of FIG. 26. At step S801, by turning on the first and second exciting light sources 38a and 38b and inserting the shutter 36 into the optical path of the reference-light source 31, a subject is illuminated by the first and second exciting lights.

At step S802, one field of an image signal is ordered to be generated. After generating an image signal, the process proceeds to step S803, where it is determined whether are input for changing the observation mode is detected. If there is no input for changing, the process returns to step S701. Until the input for changing is detected, steps S801-S803 are repeated. If there is input for changing, the process returns to a former subroutine for the endoscope driving process.

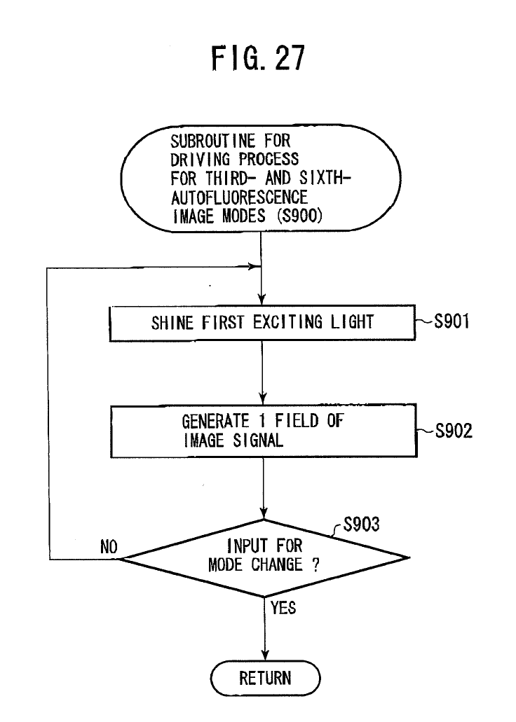

Next, a subroutine for the driving process for the third- and sixth-autofluorescence image mode (S900) is explained using the flowchart of FIG. 27. At step S701, by turning on the first exciting light source 38a, turning off the second exciting light source 38b, and inserting the shutter 36 into the optical path of the reference-light source 31, a subject is illuminated by the first exciting light.

At step S902, one field of an image signal is ordered to be generated. After generating an image signal, the process proceeds to step, S903, where it is determined whether an input for changing the observation mode is detected. If there is no input for changing, the process returns to step S901. Until the input for changing is detected, steps S901-S903 are repeated. If there is input for changing, the process returns to a former subroutine for the endoscope driving process.

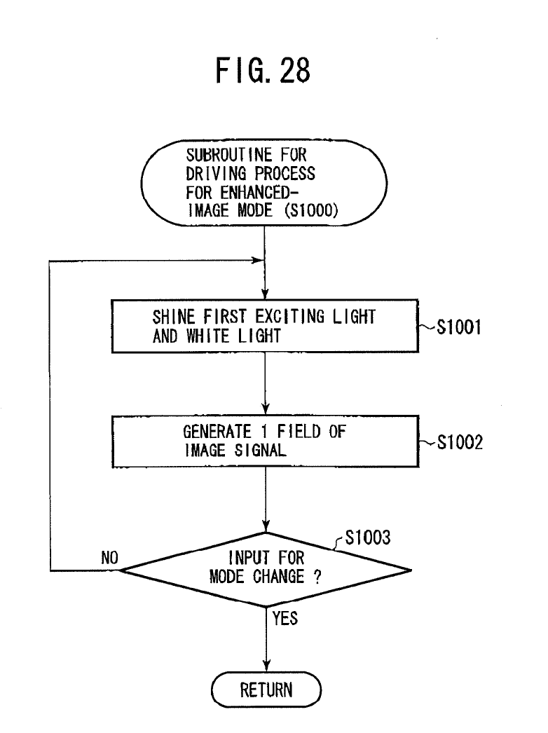

Next, a subroutine for the driving process for the enhanced-image mode (S1000) is explained using the flowchart of FIG. 28. At step S1001, by turning on the first exciting light source 38a, turning off the second exciting light source 38b, and removing the shutter 36 from the optical path of the reference-light source 31, a subject is illuminated by the first exciting light and the white light.

At step S1002, one field of an image signal is ordered to be generated. After generating an imago signal, the process proceeds to step S1003, where it is determined whether an input for changing the observation mode is detected. If there is no input for changing, the process returns to step S1001. Until the input for changing is detected, steps S1001-S1003 are repeated. If there is input for changing, the process returns to a former subroutine for the endoscope driving process.

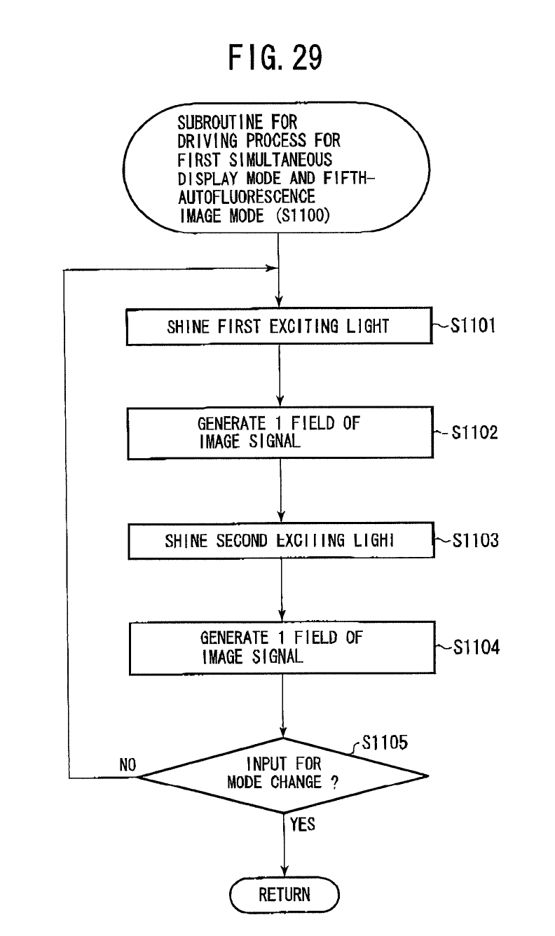

Next, a subroutine for the driving process for the first simultaneous display mode and the sixth-autofluorescence image mode (S1100) is explained using the flowchart of FIG. 29. At step S1101, by turning on the first exciting light source 38a, turning off the second exciting light source 38b, and inserting the shutter 36 into the optical path of the reference-light source 31, a subject is illuminated by the first exciting light.

At step S1102, one field of an image signal is ordered to be generated. After generating an image signal, the process proceeds to step S1103.

At step S1103, by turning off the first exciting light source 38a, turning on the second exciting light source 38b, and inserting the shutter 36 into the optical path of the reference-light source 31, a subject is illuminated by the second exciting light.

At step S1104, one field of an image signal is ordered to be generated. After generating an image signal, the process proceeds to step S1105, where it is determined whether an input for changing the observation mode is detected. If there is no input for changing, the process returns to step S1101. Until the input for changing is detected, steps S1101-S1105 are repeated. If there is input for changing, the process returns to a former subroutine for the endoscope driving process.

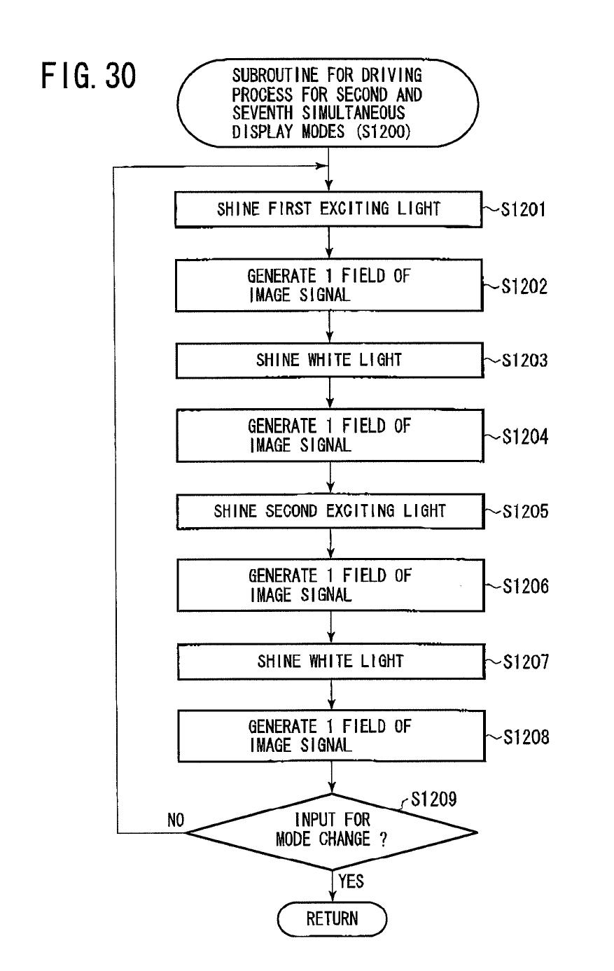

Next, a subroutine for the driving process for the second and seventh simultaneous display modes (S1200) is explained using the flowchart of FIG. 30. At step S1201, by turning on the first exciting light source 38a, turning off the second exciting light source 38b, and inserting the shutter 36 into the optical path of the reference-light source 31, a subject is illuminated by the first exciting light.

At step S1202, one field of an image signal is ordered to be generated. After generating an image signal, the process proceeds to step S1203. At step S1203, by turning off the first and second exciting light sources 38a and 38b and removing the shutter 36 from the optical path of the reference-light source 31, a subject is illuminated by the white light.

At step S1204, one field of an image signal is ordered to be generated. After generating an image signal, the process proceeds to step S1205. At step S1205, by turning off the first exciting light source 38a, turning on the second exciting light source 38b, and inserting the shutter 36 into the optical path of the reference-light source 31, a subject is illuminated by the second exciting light.

At step S1206, one field of an image signal is ordered to be generated. After generating an image signal, the process proceeds to step S1207. At step S1207, by turning off the first and second exciting light sources 38a and 38b and removing the shutter 36 from the optical path of the reference-light source 31, a subject is illuminated by the white light.

At step S1208, one field of an image signal is ordered to be generated. After generating an image signal, the process proceeds to step S1209, where it is determined whether an input for changing the observation mode is detected. If there is no input for changing, the process returns to step S1201. Until the input for changing is detected, steps S1201-S1209 are repeated. If there is input for changing, the process returns to a former subroutine for the endoscope driving process.

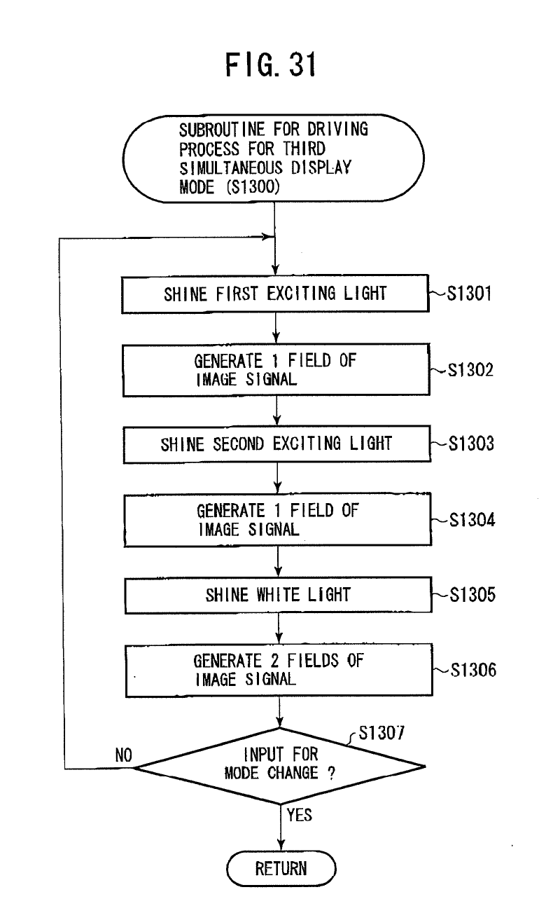

Next, a subroutine for the driving process for the third simultaneous display mode (S1300) is explained using the flowchart of FIG. 31. At step S1301, by turning on the first exciting light source 38a, turning off the second exciting light source 38b, and inserting the shutter 36 into the optical path of the reference-light source 31, a subject is illuminated by the first exciting light.

At step S1302, one field of an image signal is ordered to be generated. After generating an image signal, the process proceeds to step S1303. At step S1303, by turning off the first exciting light source 38a, turning on the second exciting light source 38b, and inserting the shutter 36 into the optical path of the reference-light source 31, a subject is illuminated by the second exciting light.

At step S1304, one field of an image signal is ordered to be generated. After generating an image signal, the process proceeds to step S1305. At step S1305, by turning off the first and second exciting light sources 38a and 38b and removing the shutter 36 from the optical path of the reference-light source 31, a subject is illuminated by the white light.

At step S1306, consecutive two fields of an image signals are ordered to be generated. After generating an image signal, the process proceeds to step S1307, where it is determined whether an input for changing the observation mode is detected. If there is no input for changing, the process returns to step S1301. Until the input for changing is detected, steps S1301-S1307 are repeated. If there is input for changing, the process returns to a former subroutine for the endoscope driving process.

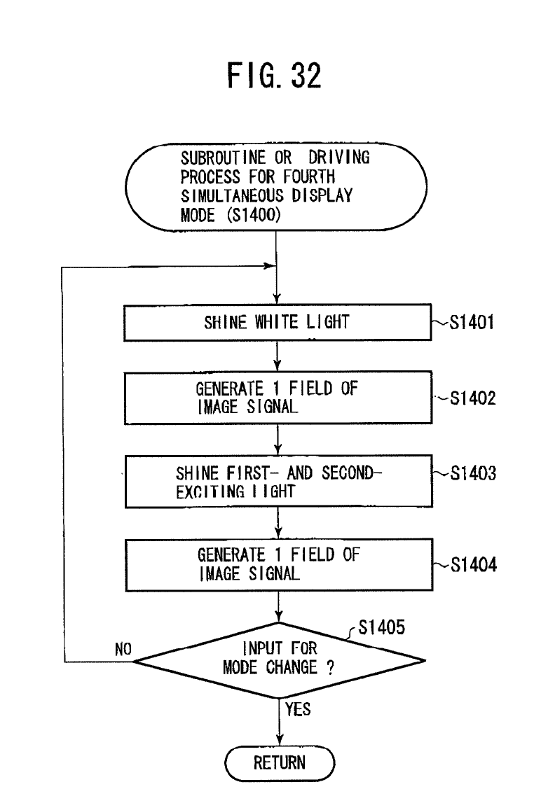

Next, a subroutine for the driving process for the fourth simultaneous display mode (S1400) is explained using the flowchart of FIG. 32. At step S1401, by turning off the first and second exciting light sources 38a and 38b and removing the shutter 36 from the optical path of the reference-light source 31, a subject is illuminated by the white light.

At step S1402, one field of an image signal is ordered to be generated. After generating an image signal, the process proceeds to step S1403. At step S1403, by turning on the first and second exciting light sources 38a and 38b and inserting the shutter 36 into the optical path of the reference-light source 31, a subject is illuminated by the first and second exciting lights.

At step S1404, one field of an image signal is ordered to be generated. After generating an image signal, the process proceeds to step S1405, where it is determined whether an input for changing the observation mode is detected. If there is no input for changing, the process returns to step S1401. Until the input for changing is detected, steps S1401-S1405 are repeated. If there is input for changing, the process returns to a former subroutine for the endoscope driving process.

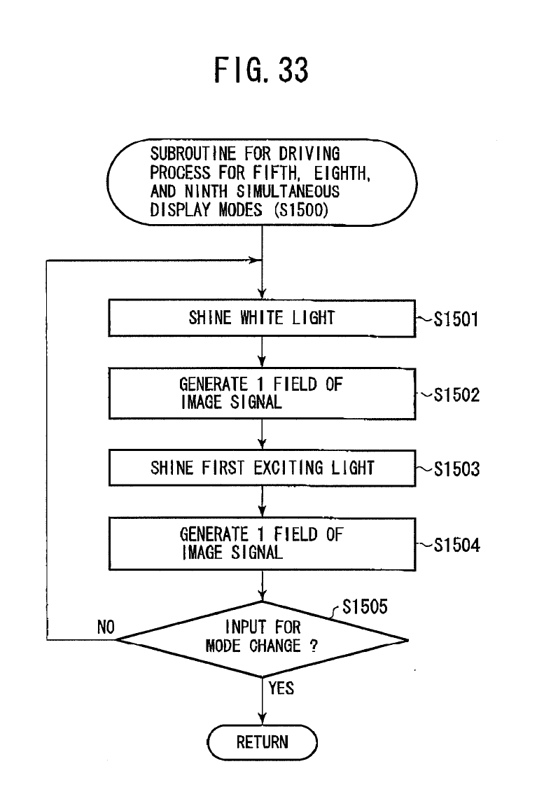

Next, a subroutine for the driving process for the fifth, eighth, and ninth simultaneous display modes (S1500) is explained using the flowchart of FIG. 33. At step S1501, by turning off the first and second exciting light sources 38a and 38b and removing the shutter 36 from the optical path of the reference-light source 31, a subject is illuminated by the white light.

At step S1502, one field of an image signal is ordered to be generated. After generating an image signal, the process proceeds to step S1503. At step S1503, by turning on the first exciting light source 38a, turning off the second exciting light source 38b, and inserting the shutter 36 into the optical path of the reference-light source 31, a subject is illuminated by the first exciting light.

At step S1504, one field of an image signal is ordered to be generated. After generating an image signal, the process proceeds to step S1505, where it is determined whether an input for changing the observation mode is detected. If there is no input for changing, the process returns to step S1501. Until the input for changing is detected, steps S1501-S1505 are repeated. If there is input for changing, the process returns to a former subroutine for the endoscope driving process.

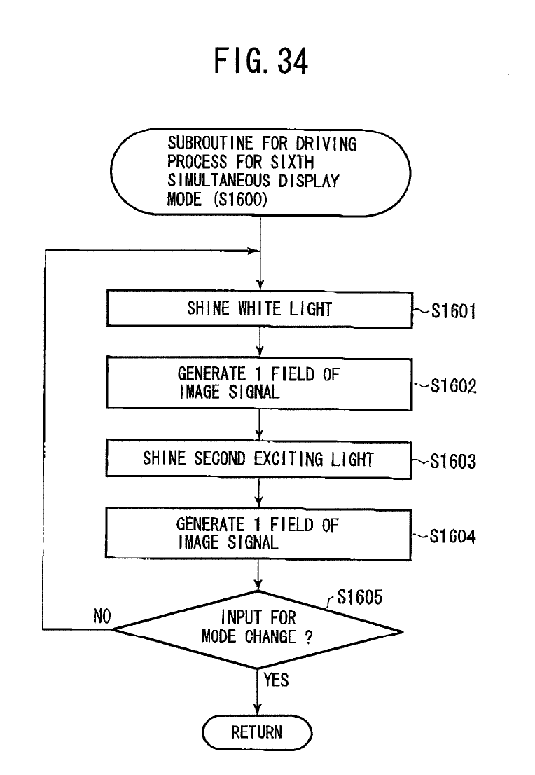

Next, a subroutine for the driving process for the sixth simultaneous display mode (S1600) is explained using the flowchart of FIG. 34. At step S1601, by turning off the first and second exciting light sources 38a and 38b and removing the shutter 36 from the optical path of the reference-light source 31, a subject is illuminated by the white light.

At step S1602, one field of an imago signal is ordered to be generated. After generating an image signal, the process proceeds to step S1603. At step S1603, by turning off the first exciting light source 38a, turning on the second exciting light source 38b, and inserting the shutter 36 into the optical path of the reference-light source 31, a subject is illuminated by the second exciting light.

At step S1604, one field of an image signal is ordered to be generated. After generating an image signal, the process proceeds to step S1605, where it is determined whether an input for changing the observation mode is detected. If there is no input for changing, the process returns to step S1601. Until the input for changing is detected, steps S1601-S1605 are repeated. If there is input for changing, the process returns to a former subroutine for the endoscope driving process.

In the above embodiment, various kinds of images to help diagnosis, for example, some kinds of autofluorescence images having a different wavelength band, an enhanced image, a full-color enhanced image, and so on, can be displayed.

Furthermore, in the above embodiment, the light-source unit 30 can be used for an electronic endoscope of general purpose. Even if the light-source unit 30 is used for an electronic endoscope without an exciting-light cut-off filter, the normal image and the first enhanced image can be displayed. Of course, if the light-source unit 30 is used for an electronic endoscope having an exciting-light cut-off filter in front of a receiving surface of an imaging devices various kinds of autofluorescence images adequate for a subject can be displayed.

The light-source unit 30 is used for an electronic endoscope in the above embodiment. However, it can be used for a fiberscope.

Although the embodiments of the present invention have been described herein with reference to the accompanying drawings, obviously many modifications and changes may be made by those skilled in this art without departing from the scope of the invention.

The present disclosure relates to subject matter contained in Japanese Patent Application No. 2007-105577 (filed on Apr. 13, 2007), which is expressly incorporated herein, by reference, in its entirety.

1. An autofluorescence endoscope system, comprising:
a first exciting light source that emits first exciting light, the wavelength of said first exciting light ranging in a first band, said first exciting light making an organ autofluoresce;
a second exciting light source that emits second exciting light, the wavelength of said second exciting light ranging in a second band, the wavelength of said second band being longer than that of said first band, said second exciting light making an organ autofluoresce;
an exciting-light cut-off filter that attenuates a light component at least of said first or second bands from an optical image of a subject illuminated by said first and second exciting lights;
an imaging device that captures an optical image of said required subject passing said exciting-light cut-off filter, said imaging device generating an image signal corresponding to a captured optical image;
a light-source controller that controls said first and second exciting light sources; and
an imaging device driver that drives said imaging device.

2. An autofluorescence endoscope system according to claim 1, wherein said exciting-light cut-off filter is a trap filter which attenuates exciting light of said second band.

3. An autofluorescence endoscope system according to claim 2, further comprising:
a reference-light source that emits white light to illuminate said required subject; and
a cut-off filter that attenuates light components of a predetermined band in said white light, said predetermined band including said first and second bands;
said light-source controller ordering said first exciting light source and said reference-light source to simultaneously emit said first exciting light and white light, respectively; and
said imaging device driver ordering said imaging device to capture an optical image passing said exciting-light cut-off filter when said first exciting light and said reference light are simultaneously emitted.

4. An autofluorescence endoscope system according to claim 3, therein,
said light-source controller orders said first and second exciting light sources and said reference-light source to alternately repeat simultaneous emission of said first exciting light and said reference light and emission of said second exciting light, and
said imaging device driver orders said imaging device to generate one field of an image signal when said first exciting light and said reference light are emitted simultaneously and when said second exciting light is emitted.

5. An autofluorescence endoscope system according to claim 2, further comprising a reference-light source that emits white light to illuminate said subject,
said light-source controller ordering said first exciting light source and said reference-light source to alternately and repeatedly emit said first exciting light and said white light,
said imaging device driver ordering said imaging device to generate one field of an image signal when said first exciting light is emitted and when said white light is emitted.

6. An autofluorescence endoscope system according to claim 2, further comprising a reference-light source that emits white light to illuminate said subject,
said light-source controller ordering said first exciting light source and said reference-light source to alternately and repeatedly emit said first exciting light and said white light,
said imaging device driver ordering said imaging device to generate one field and two fields of an image signal when said first exciting light is emitted and when said white light is emitted, respectively.

7. An autofluorescence endoscope system according to claim 2, wherein said light-source controller orders said second exciting light source to emit said second exciting light, said imaging device driver orders said imaging device to capture an optical image passing said exciting-light cut-off filter when said second exciting light is emitted.

8. An autofluorescence endoscope system according to claim 1, wherein said exciting-light cut-off filter attenuates exciting light whose wavelength in equal to or less than said second band.

9. An autofluorescence endoscope system according to claim 8, wherein said light-source controller orders said first and second exciting light sources to simultaneously emit said first and second exciting light, said imaging device driver orders said imaging device to capture an optical image passing said exciting-light out-off filter when said first and second exciting light are simultaneously emitted.

10. An autofluorescence endoscope system according to claim 8, wherein said light-source controller orders said second exciting light source to emit said second exciting light, said imaging device driver ordering said imaging device to capture an optical image passing said exciting-light cut-off filter when said second exciting light is emitted.

11. An autofluorescence endoscope system according to claim 8, wherein said light-source controller ordering said first and second exciting light sources to alternately and repeatedly emit said first and second exciting lights said imaging device driver ordering said imaging device to generate one field of an image signal when said first exciting light is emitted and when said second exciting light is emitted.

12. An autofluorescence endoscope system according to claim 11, further comprising an image signal processor that generates a synthesized autofluorescence image signal based on first and second autofluorescence image signals, said first and second autofluorescence image signals being generated by capturing an optical image when said first and second exciting lights are separately emitted.

13. An autofluorescence endoscope system according to claim 1, wherein said exciting-light cut-off filter attenuates exciting light whose wavelength is equal to or less than said first band.

14. An autofluorescence endoscope system according to claim 13, wherein said light-source controller ordering said first exciting light source to emit said first exciting light, said imaging device driver ordering said imaging device to capture an optical image passing said exciting-light cut-off filter when said first exciting light is emitted.

15. A light-source unit that supplies light to illuminate a subject at a head end of an insert tube of an endoscope, said light-source unit comprising:
a first exciting light source that emits first exciting light, the wavelength of said first exciting light ranging in a first band, said first exciting light making an organ autofluoresce;
a second exciting light source that emits second exciting light, the wavelength of said second exciting light ranging in a second band, the wavelength of said second band being longer than that of said first band, said second exciting light making an organ autofluoresce;
a receiver that receives property information concerning the kind of said endoscope from a memory mounted in said endoscope; and
a light-source controller that controls said first and second exciting light sources based on said property information.

16. A light-source unit according to claim 15, further comprising:
a reference-light source that emits white light to illuminate said subject; and
a cut-off filter that attenuates a light component of predetermined band in said white light, said predetermined band including said first and second bands;
said light-source controller ordering said first exciting light source and said reference-light source to simultaneously emit said first exciting light and white light, respectively, when said property information indicates that an imaging device mounted in said endoscope processor is covered with a trap filter which attenuates exciting light of said second band.

17. A light-source unit according to claim 16, wherein,
said light-source controller orders said first and second exciting light sources and said reference-light source to alternately repeat simultaneous emission of said first exciting light and said reference light and emission of said second exciting light.

18. A light-source unit according to claim 15, further comprising a reference-light source that emits white light to illuminate said subject,
said light-source controller ordering said first exciting light source and said reference-light source to alternately and repeatedly emit said first exciting light and white light when said property information indicates that an imaging device mounted in said endoscope processor is covered with a trap filter which attenuates exciting light of said second band.

19. A light-source unit according to claim 15, wherein said light-source controller orders said first and second exciting light sources to simultaneously emit said first and second exciting light when said property information indicates that an imaging device mounted in said endoscope processor is covered with an exciting-light cut-off filter which attenuates exciting light whose wavelength is equal to or less than said second band.

20. A light-source unit according to claim 15, wherein said light-source controller orders said first and second exciting light sources to alternately and repeatedly emit said first and second exciting light when said property information indicates that an imaging device mounted in said endoscope processor is covered with an exciting-light cut-off filter which attenuates exciting light whose wavelength is equal to or less than said second band.
